(19) United States
(12) Patent Application Publication
(10) Pub. No.: US 2024/0354946 A1
(43) Pub. Date: Oct. 24, 2024

(54) SYSTEMS AND METHODS FOR PET IMAGING ANALYSIS FOR BIOLOGY-GUIDED RADIOTHERAPY

(71) Applicant: RefleXion Medical, Inc., Hayward, CA (US)

(72) Inventors: Linxi SHI, Los Altos, CA (US); Angela Jane DA SILVA, Danville, CA (US); Maksat HAYTMYRADOV, Hayward, CA (US); Peter Demetri OLCOTT, Los Gatos, CA (US); George Andrew ZDASIUK, Portola Valley, CA (US)

(21) Appl. No.: 18/641,343

(22) Filed: Apr. 20, 2024

Related U.S. Application Data
(63) Continuation of application No. PCT/US2022/078511, filed on Oct. 21, 2022.
(60) Provisional application No. 63/392,446, filed on Jul. 26, 2022.
(60) Provisional application No. 63/270,404, filed on Oct. 21, 2021.

(51) Int. Cl. G06T 7/00; G16H 20/40; G16H 50/20

(52) U.S. Cl. CPC G06T 7/0012; G16H 20/40; G16H 50/20; G06T 2207/10081; G06T 2207/10104; G06T 2207/30096

(57) ABSTRACT

Disclosed herein are methods for determining suitability of biology-guided radiotherapy (BgRT). These methods may include converting diagnostic positron emission tomography (PET) imaging data to simulated imaging data consistent with images obtained using PET detectors of a BgRT radiotherapy system. The simulated imaging data may be used to evaluate the suitability of BgRT by evaluating a first metric indicating a contrast noise ratio for a tumor, a second metric indicating a PET tracer activity concentration, and a third metric indicating a radiation dose to the tumor. Also disclosed herein are methods for generating synthetic or simulated list mode LOR data from one or more PET images. The synthetic or simulated list mode data may be used for testing BgRT algorithms and/or determining whether BgRT is suitable for a patient.

### CROSS-REFERENCE TO RELATED APPLICATIONS

This application is a continuation of International Patent Application No. PCT/US2022/078511, filed Oct. 21, 2022, which claims priority to U.S. Provisional Patent Application Ser. No. 63/270,404 filed Oct. 21, 2021, and U.S. Provisional Patent Application Ser. No. 63/392,446 filed Jul. 26, 2022, the disclosures of which are hereby incorporated by reference in their entirety.

### TECHNICAL FIELD

Disclosed herein are systems and methods for determining suitability of biology-guided radiotherapy. Also disclosed herein are methods for converting a PET image into simulated list mode lines-of-response (LOR) data.

### BACKGROUND

Biology-guided radiotherapy (BgRT) uses PET emissions to guide radiotherapy delivery in real-time. BgRT allows radiation dose delivery based on the collection and processing of positron emission tomography (PET) data from a positron-emitting radiotracer (such as 18-F fluorodeoxyglucose or FDG). Tumors uptake the tracer to a greater extent than healthy cells and emit positrons that annihilate with nearby electrons to generate a line-of-response (LOR), which is a pair of nearly co-linear 511 keV photons that travel in opposite directions from the annihilation event. PET detectors sense these LORs which may provide information about the location of the tumor. In this way, BgRT utilizes radiotracer uptake for targeting, tracking, and adjusting dose delivery in real-time to account for target motion.

The suitability of using BgRT for a patient may depend on a variety of factors, such as a type of cancer, a location of a tumor, a size of the tumor, how well the tumor absorbs the tracer, and various combinations of these factors. Thus, for successful application of BgRT, it is important to establish a process for determining the suitability of BgRT. Accordingly, systems and methods for making such a determination would be desirable.

### SUMMARY

Disclosed herein are methods for determining suitability of biology-guided radiotherapy (BgRT). In one variation, the method may include converting diagnostic positron emission tomography (PET) imaging data to simulated imaging data consistent with images obtained using PET detectors of a BgRT radiotherapy system. The simulated imaging data and the diagnostic PET imaging data may represent a PET signal from a tracer. Further, based on the simulated imaging data, the method may include determining: a first metric indicating a contrast noise ratio for a tumor, a second metric indicating a PET tracer activity concentration, and a third metric indicating an estimated radiation dose to the tumor. Additionally, the method may include determining the suitability of using the BgRT based on the first, the second, and the third metric. Some variations may comprise determining that BgRT is suitable if a value of at least one of the first metric value, the second metric value, and the third metric value is within the range of acceptable values.

Also disclosed herein is a method of converting diagnostic PET imaging data to simulated imaging data consistent with images obtained during a BgRT session. In one variation, the method includes calibrating the sensitivity of the PET detectors of a BgRT radiotherapy system relative to sensitivity of a PET imaging system that was used for obtaining PET image data, converting the sinogram to expected counts per sinogram-bin, and modifying the expected counts based on parameters of the BgRT radiotherapy system, wherein the parameters include at least the sensitivity of the PET detectors of a BgRT radiotherapy system, subject to an efficiency of the PET detectors of the BgRT radiotherapy system and a time used by the BgRT radiotherapy system for collecting data. Further, the method may include modifying the expected counts by adding noise modeled by Poisson statistics and reconstructing the simulated imaging data based on the modified expected counts.

Disclosed herein is a method for determining suitability of biology-guided radiotherapy (BgRT). The method includes converting diagnostic positron emission tomography (PET) imaging data to simulated imaging data consistent with images obtained using PET detectors of a BgRT radiotherapy system, the simulated imaging data and the diagnostic PET imaging data representing a PET signal from a tracer. Further, based on the simulated imaging data, the method includes determining a first metric indicating a contrast noise ratio for a tumor, a second metric indicating a PET tracer activity concentration, and a third metric indicating a radiation dose to the tumor. The method also includes determining the suitability of using the BgRT based on the first, the second, and the third metric. Some variations may comprise calculating the first metric, the second metric and the third metric using the simulated imaging data. The method may comprise determining that BgRT is suitable if a value of at least one of the first metric value, the second metric value, and the third metric value is within the range of acceptable values.

In some variations, the method includes obtaining additional diagnostic PET imaging data, converting the additional diagnostic PET imaging data to new simulated imaging data consistent with images obtained when performing BgRT (alternatively, or additionally, consistent with imaging data obtained using PET detectors of a BgRT radiotherapy system), and based on the new simulated imaging data, calculating a new first metric value indicating a contrast normalization signal for a tumor, calculating a new second metric value indicating a PET tracer activity concentration, and calculating a new third metric value indicating a radiation dose for a volume of the tumor. The method also includes determining the suitability of using the BgRT based on the new first, the new second, and the new third metric. The method may comprise determining that BgRT is suitable if a value of at least one of the new first metric value, the new second metric value, and the new third metric value is within the range of acceptable values.

In some variations, the method includes determining the suitability of using the BgRT based on a difference between the new first metric value and the first metric value, the new second metric value and the second metric value, and the new third metric value and the third metric value.

In some variations of the method, the additional diagnostic PET imaging data is obtained prior to performing a BgRT treatment, the BgRT treatment not forming part of the method.

In some variations of the method, the first metric is determined as a difference between a mean signal in a target region <T_{S}> and a mean signal in a background region <Bg> divided by a variance of the signal σ_{Bg} in the background region (<T_{S}>−<Bg>)/σ_{Bg}.

In some variations of the method, where the signal in a target region Tₛ is calculated in a portion of a clinical target volume (and/or a planning target volume) in which a value of a PET signal is less than a target threshold percent of a peak value of the PET signal as measured in the clinical target volume.

In some variations of the method, the target threshold percent is fifty percent.

In some variations of the method, the first metric is determined as median activity concentration of a target region (PTV) divided by a mean signal in a background region <Bg>: MedianAC[PTV]/<Bg>.

In some variations of the method, Bg is calculated over a shell region, the shell region being a portion of a biological targeting zone and not a part of a clinical target volume.

In some variations of the method, determining the suitability of using the BgRT comprises determining that the contrast normalization signal is above a required threshold for the signal, that the PET tracer activity concentration is above a minimal concentration threshold, and that the determined radiation dose is within a pre-defined dose range.

In some variations of the method, the pre-defined dose range is represented by an upper dose-volume histogram (DVH) curve and a lower DVH curve of a bounded DVH.

In some variations of the method, when the suitability of using BgRT is not indicated, the method includes obtaining an additional diagnostic PET imaging data using a different type of PET tracer than a type of PET tracer that is used for obtaining the diagnostic PET imaging data. In some variations, the additional diagnostic PET imaging data may be previously generated or acquired in a prior imaging session, and stored in a controller memory. In another variation, the different type of PET tracer may be introduced to a patient prior to performing the method. Its introduction therefore does not form part of the method.

In some variations of the method, the first metric is further verified by obtaining visual representation of the tumor using CT imaging.

In some variations of the method, the radiation dose comprises a function determining acceptable radiation doses for a given volume fraction of a tumor tissue.

In some variations, the method further includes converting the simulated imaging data to single line-of-response (LOR) data between a pair of detector elements.

In some variations, the method further includes generating a BgRT plan, the BgRT plan including an identified target region, and firing filters that convert PET imaging data into a radiation fluence map that results in the prescribed dose being delivered to the identified tissue.

In some variations of the method, converting the diagnostic PET imaging data to the simulated imaging data consistent with images obtained using PET detectors of a BgRT radiotherapy system (e.g., images obtained when performing BgRT) includes calibrating sensitivity of the PET detectors of the BgRT radiotherapy system, generating a sinogram based on the PET imaging data, wherein the generating includes at least correcting for an attenuation using computer tomography (CT) data, converting the sinogram to expected counts per sinogram-bin, modifying the expected counts based on parameters of the BgRT radiotherapy system, modifying the expected counts by adding noise modeled by Poisson statistics, and reconstructing the simulated imaging data based on the modified expected counts. The parameters may include at least the sensitivity of the BgRT radiotherapy system subject to an efficiency of the BgRT radiotherapy system and a time used by the BgRT radiotherapy system for collecting data.

In some variations of the method, converting the diagnostic PET imaging data to the simulated imaging data consistent with images obtained using PET detectors of a BgRT radiotherapy system (e.g., images obtained when performing BgRT) further comprises determining the sinogram based on the PET imaging data by modeling photon scatter in a PET detector scintillator.

In some variations of the method, the noise modeled by Poisson statistics is based on random coincidences.

In some variations of the method, the noise modeled by Poisson statistics is based on random detection events.

In some variations of the method, the sinogram is corrected by truncating the sinogram to a field of view that includes the tumor.

In some variations of the method, the target field of view has a size of 50 centimeters.

Further, disclosed herein is a method of converting a diagnostic PET imaging data to a simulated imaging data consistent with images obtained using PET detectors of a BgRT radiotherapy system (e.g., obtained during BgRT session), the method comprising calibrating sensitivity of the PET detectors of a BgRT radiotherapy system, converting the sinogram to expected counts per sinogram-bin, modifying the expected counts based on parameters of the BgRT radiotherapy system, wherein the parameters include at least the sensitivity of the BgRT radiotherapy system subject to an efficiency of the BgRT radiotherapy system and a time used by the BgRT radiotherapy system for collecting data, modifying the expected counts by adding noise modeled by Poisson statistics, and reconstructing the simulated imaging data based on the modified expected counts.

In some variations of the method, the expected counts are converted to a second sinogram for the simulated imaging data, and the simulated imaging data is reconstructed from the second sinogram via filtered backprojection.

In some variations of the method, the filtered backprojection utilizes empirical data from the BgRT radiotherapy system.

Further, disclosed herein is a method for simulating a second PET image based on a first PET image. The method includes converting a first PET image of a target region into a sinogram, generating list mode data from the sinogram by sampling LORs from the sinogram to include noise characteristics and component characteristics of PET detectors of a PET imaging system and serializing the sampled LORs into a list mode LOR data, with each sampled LOR having a corresponding time stamp, and generating a second PET image of the target region by filtering and backprojecting the list mode LOR data.

In some variations of the method, the noise characteristics of the PET detectors of the PET imaging system include at least one of photon scatter noise, Poisson noise, attenuation effects, and random photon coincidences.

In some variations of the method, the component characteristics of PET detectors comprise at least one of detection efficiency, detector crystal width, detector acquisition rate, detector resolution, and detector time resolution.

In some variations of the method, the list mode LOR data include time stamps corresponding to individual LORs.

In some variations of the method, the first PET image is acquired using a first PET imaging system that includes one of a three-dimensional (3D) or a four-dimensional (4D) PET.

In some variations of the method, the first PET image is a 3D or 4D computer-generated PET image of a virtual phantom.

In some variations of the method, the first PET image is acquired for a portion of an anatomy.

In some variations of the method, a location of the target region changes with time along a motion trajectory with physiological functions of the anatomy, and a plurality of PET images are obtained for different points in time.

In some variations of the method, the first PET image is an average of a plurality of PET images acquired over time.

In some variations, the method further includes grouping each of the plurality of PET images into PET image phases based on the location of the target region along the motion trajectory, and for each phase, selecting from the corresponding PET image phase, a representative PET image as the first PET image.

In some variations, the method further includes saving the generated second PET image as a data record associated with the corresponding PET image phase.

In some variations of the method, the representative PET image is an average of PET images from the corresponding PET image phase.

In some variations of the method, the motion trajectory of the target region is a breathing motion trajectory.

In some variations, the method further includes reconstructing a sinogram for each phase derived from the list mode LOR data.

In some variations of the method, the motion trajectory of the target region is a peristaltic motion trajectory.

In some variations of the method, the motion trajectory of the target region is a user-defined motion trajectory.

In some variations of the method, the list mode LOR data comprises LORs, each LOR having a corresponding detection event time stamp and associated coordinates of detectors for detecting the LOR.

Further, disclosed herein is a method for converting a PET image into simulated list mode lines-of-response (LOR) data. The method includes determining planning scan parameters for a target region in a PET image acquired using a first PET imaging system, determining biology-guided radiotherapy BgRT system parameters, generating a sinogram from the PET image for each beam station based on the planning scan parameters and the BgRT system parameters, converting the sinogram for each beam station to a second sinogram of individual lines-of-response (LORs) using a pre-calibrated scaling factor, modifying the second sinogram for each beam station to include selected artifacts for a second PET imaging system, and generating (e.g., for each beam station) a list mode LOR data by sampling LORs from the second sinogram.

In some variations of the method, for each sampled LOR a time stamp is sampled using inverse cumulative exponential density function.

In some variations of the method, the planning scan parameters include at least one of: beam station locations, beam station dwell time, number of gantry revolutions per beam station, number of beam stations, and number of couch passes through a therapeutic irradiation plane.

In some variations of the method, the BgRT system parameters include at least one of: PET detector geometry, detection efficiency, detector crystal width, detector acquisition rate, detector resolution, and detector time resolution.

In some variations of the method, the selected artifacts include at least one of: photon scatter noise, Poisson noise, attenuation effects, and random photon coincidences.

In some variations of the method, a location of the target region changes with time along a motion trajectory with physiological functions of the anatomy, and wherein a plurality of PET images are obtained for different points in time.

In some variations of the method, the motion trajectory of the target region is a breathing motion trajectory.

In some variations of the method, the motion trajectory of the target region is a peristaltic motion trajectory.

In some variations of the method, the motion trajectory of the target region is a user-defined motion trajectory.

Further, disclosed herein is a method for converting a PET image into simulated lines-of-responses (LORs). The method includes generating a sinogram from a PET image of a target region and generating a list mode LOR data based on the generated sinogram, wherein the list mode LOR data comprises a list of simulated LORs, and wherein the list of the simulated LORs is generated based on a sample of emission events (e.g., a random sample of emission events).

In some variations of the method, the sample of emission events is generated using an inverse transform sampling method, and the inverse transform sampling method is based on a cumulative density function characterizing emission events represented by the generated sinogram.

In some variations of the method, the inverse transform sampling method uses uniformly distributed random numbers on an interval of zero to one representing a likelihood of the emission event, and for each random number an inverse of cumulative density function is computed to determine a sinogram bin and an associated simulated LOR.

In some variations, the method includes modifying the simulated LORs to include noise characteristics and component characteristics of PET detectors of a PET imaging system.

In some variations, the method includes using a filtered back-projection method and the list mode LOR data to generate a simulated PET image of the target region.

In some variations of the method, the list mode LOR data includes a time stamp data [ts], a time difference between two recorded emission events [dt], and a position of a gantry [lpos].

In some variations of the method, a PET imaging system (e.g., the PET imaging system of a BgRT radiotherapy system) may comprise a first detecting arc and a second detecting arc that are rotatable about the target region. Further, the simulated LORs are computed for each time interval corresponding to angular position of the first and the second detecting arc, and the simulated LORs associated with emission events not detected by the first and the second detecting arc are discarded.

In some variations of the method, including noise characteristics and component characteristics of the PET detectors includes accounting for the scattering at the PET detectors.

In some variations of the method, including noise characteristics and component characteristics of the PET detectors includes accounting for the PET detectors efficiency.

In some variations of the method, including noise characteristics and component characteristics of the PET detectors includes accounting for the lower photon capture at an edge of the PET detectors field of view PET detectors efficiency.

In some variations, the method comprises including attenuation characteristics of a media forming the target region.

In some variations of the method, the attenuation characteristics are determined based on a computer tomography scan of the target region.

In some variations, the method includes generating a radiotherapy treatment plan for the target region based on the list mode LOR data.

In some variations, for various methods described above, the list mode data is generated for each beam station.

Also disclosed herein is a method for simulating a second PET image based on a first PET image, for example, a time-of-flight (TOF) PET image. The method may comprise converting a first PET image of a target region into a plot that comprises a number of positron annihilation photon emission events for each pixel in a PET image, sampling emission events from the plot to include noise characteristics and component characteristics a PET imaging system, generating list mode data from the plot by serializing the sampled emission events by assigning a time stamp to each sampled emission event, and generating a second PET image of the target region using the list mode data by plotting an intensity level at every pixel that correlates with the number of emission events at that pixel. The noise characteristics of PET detectors of the PET imaging system may include at least one of: photon scatter noise, Poisson noise, attenuation effects, and random photon coincidences. The component characteristics of PET detectors may include at least one of: detection efficiency, detector crystal width, detector acquisition rate, detector resolution, and detector time resolution. In some variations, the list mode data may include time stamps corresponding to individual LORs from the sampled emission events. In some variations, the first PET image may include a plurality of PET images acquired of the target region over time. For example, the location of the target region may change with time along a motion trajectory, and the plurality of PET images may be obtained for different points in time. For example, motion trajectory of the target region may be a breathing motion trajectory, and/or a peristaltic motion trajectory, and/or any user-defined motion trajectory. In some variations, the method may further include grouping each of the plurality of PET images into PET image phases based on the location of the target region along the motion trajectory, and for each phase, selecting a representative PET image as the first PET image and generating list mode data for each phase by converting the PET image into a plot comprising a number of positron annihilation photon emission events for each pixel, sampling emission events from the plot, and serializing the sampled emission events by assigning a time stamp to each sampled emission event. Optionally, the method may comprise generating a sinogram for each phase derived from the list mode data for that phase. In some variations, the list mode data may include a plurality of emission events, where each emission event having a corresponding detection event time stamp and associated coordinates of detectors for detecting an LOR for each emission event.

Also described herein is a method for generating synthetic LORs from a PET image. One variation of a method may include sampling positron annihilation photon emission events from a PET image, selecting a detection angle for each sampled emission event, determining an offset based on the spatial coordinates and the selected detection angle for each sampled emission event, assigning a time stamp to each sampled emission event, and generating synthetic list mode LOR data by combining the detection angle, offset, and time stamp for each emission event. The initial PET image may be a TOF PET image or any PET image where an intensity of each pixel correlates to a number of emission events having spatial coordinates that correspond to a location of that pixel. In some variations, sampling the emission events may include converting the number of emission events into a probability distribution function, determining a cumulative distribution function (CDF) and an inverse CDF, and randomly selecting emission events from the generated inverse CDF. Selecting the detection angle may include randomly selecting an angle in a range of 0 degrees to 360 degrees. The spatial coordinates of a pixel and the corresponding emission events may include coordinates in IEC-X and IEC-Z, and the offset may be determined using the IEC-X coordinate, IEC-Z coordinate, and the selected detection angle. In some variations, the method may further include determining whether an LOR corresponding to an emission event (with its spatial coordinates, selected detection angle, and determined offset) intersects with PET detectors of a PET imaging system before assigning a time stamp to the emission event. In some variations, assigning the time stamp for each emission event may include selecting time intervals between emission events according to Poisson statistics.

The foregoing general description and the following detailed description are exemplary and explanatory only and are not restrictive of the claims.

### BRIEF DESCRIPTION OF THE DRAWINGS

FIG. 1 is a block diagram representation of one variation of a radiotherapy system.

FIG. 2A is one variation of a radiotherapy system.

FIG. 2B is a perspective component view of the radiotherapy system of FIG. 2A.

FIG. 2C is a schematic view of one variation of a radiotherapy system having multiple beam stations.

FIG. 2D is a perspective view of one variation of a PET imaging system (e.g., PET imaging system of a BgRT radiotherapy system).

FIG. 2E is a cross-sectional view of the PET imaging system according to the variation of FIG. 2D.

FIG. 2F is an example of series of positron annihilation photon emission events having corresponding line-of-responses (LORs).

FIG. 2G is sinogram data point for a single emission event.

FIG. 2H is a sinogram for multiple emission events from a single volume or voxel of tissue.

FIG. 3 is an example method for determining suitability of a biology-guided radiotherapy (BgRT) procedure.

FIG. 4A is an example method for generating a second PET image of a target region based on a first PET image.

FIG. 4B are example PET images and sinograms.

FIG. 4C is an example list of parameters that affect determination of simulated imaging data.

FIGS. 5A and 5B show simulated imaging data generated by the methods disclosed herein using diagnostic PET imaging data.

FIG. 6A is a schematic representation of a biology tracking zone (BTZ) surrounded by a shell region and containing a clinical target volume (CTV) and a planning target volume (PTV).

FIG. 6B is a schematic representation of PTV and CTV as used by a traditional stereotactic body radiation therapy (SBRT).

FIG. 6C is a schematic representation of BTZ further including a motion envelope zone as used by BgRT.

FIG. 6D is a signal measurement representing a PET image.

FIG. 6E is a contour map corresponding to the signal measurement as shown in FIG. 6D.

FIG. 6F is a cross-sectional view of the signal measurement as shown in FIG. 6D.

FIG. 7 is an example method for determining if a calculated radiation dose is within an allowable clinical bounds.

FIG. 8 is a flowchart of one variation of a method for evaluating the suitability of BgRT.

FIG. 9 is an example dose volume histogram (DVH).

FIG. 10 is an example of a bounded dose volume histogram (bDVH).

FIG. 11 is another example of a bounded dose volume histogram (bDVH) further including a simulated DVH curve.

FIG. 12A is an example method for determining the suitability of BgRT treatment.

FIG. 12B is an example method of verifying suitability of BgRT treatment prior to the BgRT treatment.

FIG. 12C is an example method of verifying suitability of BgRT treatment during the BgRT treatment.

FIG. 13A is an example method of generating a second PET image from a first PET image.

FIG. 13B is an example method of generating a second PET image from a first PET image.

FIG. 14A is an example method of generating a list mode LOR data.

FIG. 14B is an example diagram describing the generation of a simulated sinogram.

FIG. 15A is an example method of serializing LORs from a sinogram to generate synthetic list mode LOR data.

FIG. 15B are example sinogram slices and sinogram bins.

FIG. 15C is an example method of generating sampling curves by creating inverse cumulative distribution functions.

FIGS. 15D and 15E are example approaches of generating cumulative distribution functions.

FIG. 15F is an example method of sampling an LOR using inverse cumulative distribution functions.

FIG. 15G depicts an example cumulative distribution function and the inverse cumulative distribution function.

FIG. 16A is an example method of generating synthetic list mode LOR data using a PET image.

FIG. 16B is an example diagram for determining an offset S for an LOR.

FIG. 16C is another example method of generating synthetic list mode LOR data using a PET image.

FIG. 16D is another example method of generating synthetic list mode LOR data using a PET image.

FIG. 17A is a histogram indicating the time interval between LOR counts that are collected as a function of time.

FIG. 17B is a probability distribution function associated with the histogram of FIG. 17A.

FIG. 18 are example of several sinograms each having a different number of LOR counts.

FIG. 19 is an example approach for separating sinograms obtained from a four-dimensional PET image into phases.

### DETAILED DESCRIPTION

Biology-guided radiation therapy (BgRT) relies on image data (e.g., imaging data, images) obtained from positron emission tomography (PET) detectors to direct radiation to the real-time location of a tumor. The PET image represents a PET signal from a tracer. For example, a PET image may be composed of a plurality of grey-scaled (or colored) pixels, with a shade (or color) of a pixel (herein, also referred to as a brightness or intensity of the pixel) corresponding to a concentration of a PET tracer in a given voxel of a tissue. For example, voxels that appear bright in a PET image, i.e., have a high intensity value, may indicate that the corresponding portion of tissue contains a high concentration of a PET tracer, while voxels that appear dimmer in a PET image, i.e., have a low intensity value, may indicate that the corresponding portion of tissue contains a low concentration of the PET tracer. In the examples described herein, the concentration of a PET tracer may also be referred to as a PET tracer activity concentration (AC), since the amount of positron emission activity in a tissue may correlate with the amount of PET tracer taken up by that tissue. The PET image may include a legend that maps colors (or gray-scale values) of the PET image to a number legend representing the concentration of the PET tracer. In some variations, a PET image may be obtained for different cross-sections of three-dimensional organs. Further, the PET image may be processed and depicted as three-dimensional surfaces and/or include contour lines representing constant concentrations of the PET tracer (e.g., iso-contours). PET images utilize tracer technology, and suitable PET tracers may include fluorodeoxyglucose (FDG). Examples of PET tracers, with their radioactive isotope in parentheses, may include acetate (C-11), choline (C-11), fluorodeoxyglucose (F-18), sodium fluoride (F-18), fluoro-ethyl-spiperone (F-18), methionine (C-11), prostate-specific membrane antigen (PSMA) (Ga-68), DOTATOC, DOTANOC, DOTATATE (Ga-68), florbetaben, florbetapir (F-18), rubidium (Rb-82) chloride, ammonia (N-13), FDDNP (F-18), Oxygen-15 labeled water, and FDOPA (F-18). Other tracers are known in the art as well. It should be appreciated, that depending on a type of cancer, a particular tracer may be selected, and when one tracer is determined not to be suitable for BgRT procedure, another tracer may be used. In some variations, one tracer may be used for diagnoses while another tracer may be used for BgRT delivery.

A PET signal from a tracer varies depending on a person, a type of cancer, and even over time, thus, a process to determine the suitability of BgRT for a patient would be useful. Disclosed herein are methods for determining the suitability of BgRT for a patient and systems corresponding to the same. In some variations, the method may include confirming that a BgRT treatment plan (herein also referred to as a BgRT plan) can be generated for a patient, and/or meets clinician requirements, and/or delivers the prescribed dose to the tumor(s), and/or is safe to the patient.

### Systems

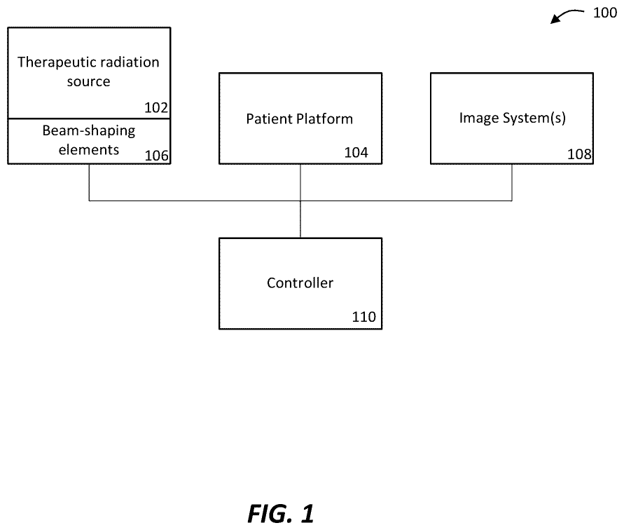

In some variations of the systems and methods described herein, a BgRT treatment may be administered using a BgRT radiotherapy system (herein also referred to as a BgRT machine). FIG. 1 depicts a functional block diagram of a variation of a radiotherapy system that may be used with one or more of the methods described herein. Radiotherapy system 100 includes one or more therapeutic radiation sources 102 and a patient platform 104. The therapeutic radiation source may include an X-ray source, electron source, proton source, and/or a neutron source. For example, a therapeutic radiation source 102 may include a linear accelerator linac, Cobalt-60 source(s), and/or an X-ray machine. The therapeutic radiation source may be movable about the patient platform so that radiation beams may be directed to a patient on the patient platform from multiple firing positions and/or firing angles. A firing position is the location of the therapeutic radiation source when it emits therapeutic radiation to the patient area of the radiotherapy system. In the example of a radiotherapy system where the therapeutic radiation source moves around the patient platform in a single-plane (e.g., moving in a circular or arc trajectory within a X-Z plane along a Y-axis), the firing position may be indicated as a firing angle. The system may continuously rotate from one firing angle to another or, alternatively, dwell at a specific firing angle for a period of time. In some variations, if the travel time from one firing position to the next is sufficiently short compared to the overall 360 degree rotation time, the dwell time at one firing position may include the travel time of the therapeutic radiation source to that firing position. In some variations, a radiotherapy system may include one or more beam-shaping elements and/or assemblies 106 that may be located in the beam path of the therapeutic radiation source. For example, a radiotherapy system may include a linac 102 and a beam-shaping assembly 106 disposed in a path of the radiation beam. The beam-shaping assembly may include one or more movable jaws and one or more collimators. At least one of the collimators may be a multi-leaf collimator (e.g., a binary multi-leaf collimator, a 2-D multi-leaf collimator, etc.). The linac and the beam-shaping assembly may be mounted on a gantry or movable support frame that includes a motion system configured to adjust the position of the linac to different firing positions about the patient platform and optionally, the beam-shaping assembly. In some variations, the linac and beam-shaping assembly may be mounted on a support structure comprising one or more robotic arms, C-arms, gimbals, and the like. The patient platform 104 may also be movable. For example, the patient platform 104 may be configured to translate a patient linearly along a single axis of motion (e.g., along the IEC-Y axis), and/or may be configured to move the patient along multiple axes of motion (e.g., 2 or more degrees of freedom, 3 or more degrees of freedom, 4 or more degrees of freedom, 5 or more degrees of freedom, etc.). In some variations, a radiotherapy system may have a 5-DOF patient platform that is configured to move along the IEC-Y axis, the IEC-X axis, the IEC-Z axis, as well as pitch and yaw. Some systems may have a 6-DOF patient platform.

In the variation shown in FIG. 1, radiotherapy system 100 also includes a controller 110 that is in communication with the therapeutic radiation source 102, beam-shaping elements or assemblies 106, patient platform 104, and one or more image systems 108 (e.g., one or more imaging systems).

Imaging systems 108 may include a PET imaging system which may be configured to obtain PET imaging data prior and/or during a BgRT therapy session. PET imaging data may also be acquired as part of a quality assurance session with a PET-avid phantom. In some variations, the PET imaging system includes a first array of PET detectors and a second array of PET detectors disposed across from the first array. The first and second arrays of PET detectors may be arranged as two arcs that are directly opposite to each other and may each have a 90° span around the patient treatment region. The PET detector arcs may not comprise an entire ring around the gantry; instead, they may be partial rings, where the therapeutic radiation source is located between the two partial rings or arcs. In some variations, the PET detectors may be time-of-flight PET detectors, which may help to identify the location of the positron annihilation event. Alternatively, or additionally, imaging systems 108 may include a CT imaging system such as a kV imaging system having a kV X-ray source and a kV detector. The kV detector may be located across the kV X-ray source. Optionally, the kV imaging system may include a dynamic multi-leaf collimator (MLC) disposed over the kV X-ray source. Additional details and examples of radiation therapy systems are described in U.S. Appl. No. U.S. application Ser. No. 15/814,222, filed Nov. 15, 2017, and PCT Appl. No. PCT/US2018/025252, filed Mar. 29, 2018, which are hereby incorporated by reference in their entireties. Alternatively, or additionally, imaging systems 108 may include a magnetic resonance imaging (MRI) system.

Controller 110 may include one or more processors and one or more machine-readable memories in communication with the one or more controller processors, which may be configured to execute or perform any of the methods described herein. The one or more machine-readable memories may store instructions to cause the processor to execute modules, processes and/or functions associated with the system, such as one or more treatment plans (e.g., BgRT treatment plans, SBRT/IMRT treatment plans, etc.), the calculation of radiation fluence maps based on treatment plan and/or clinical goals, segmentation of fluence maps into radiotherapy system instructions (e.g., that may direct the operation of the gantry, therapeutic radiation source, beam-shaping assembly, patient platform, and/or any other components of a radiotherapy system), iterative calculations for updating the location(s) of a target region, image and/or data processing associated with treatment planning and/or radiation delivery, simulating PET images from different PET imaging systems, and converting PET images into simulated or synthetic lines-of-response (LORs) that correspond with the PET images. In some variations, the memory may store treatment plan data (e.g., treatment plan firing filters, fluence map, planning images, treatment session PET pre-scan images and/or initial CT, MRI, and/or X-ray images), imaging data acquired by the imaging systems 108 before and during a treatment session, instructions for identifying the location of a target region using newly-acquired imaging data, and instructions for delivering the derived fluence map (e.g., instructions for operating the therapeutic radiation source, beam-shaping assembly and patient platform in concert). In some variations, one or more memories may also store PET metric values (e.g., metric values calculated using acquired data and/or threshold metric values), including, but not limited to one or more of contrast noise ratio (which may also be referred to as a normalized tumor signal), tracer activity concentration, and/or a radiation dose metric. These PET metric values may be used as part of a BgRT planning and treatment workflow to determine whether to proceed or to pause treatment. The controller of a radiotherapy system may be connected to other systems by wired or wireless communication channels. For example, the radiotherapy system controller may be in wired or wireless communication with a radiotherapy treatment planning system controller such that fluence maps, firing filters, initial and/or planning images (e.g., CT images, MRI images, PET images, 4-D CT images), patient data, simulated PET images, simulated LORs that correspond to a PET image, and other clinically-relevant information may be transferred from the radiotherapy treatment planning system to the radiotherapy system. The delivered radiation fluence, any dose calculations, and any clinically-relevant information and/or data acquired during the treatment session may be transferred from the radiotherapy system to the radiotherapy treatment planning system. This information may be used by the radiotherapy treatment planning system for adapting the treatment plan and/or adjusting delivery of radiation for a successive treatment session. In some variations, the radiotherapy treatment planning system may include a controller having one or more processors configured to perform the methods described herein, for example, methods for determining whether the BgRT metric values calculated from patient PET images meet threshold values that indicate BgRT may be appropriate. The radiotherapy treatment planning system may include one or more memories that store diagnostic PET images, simulated PET images, simulated LORs for a corresponding PET image, any of the BgRT metrics for any PET images, thresholds for the BgRT metrics, and the like.

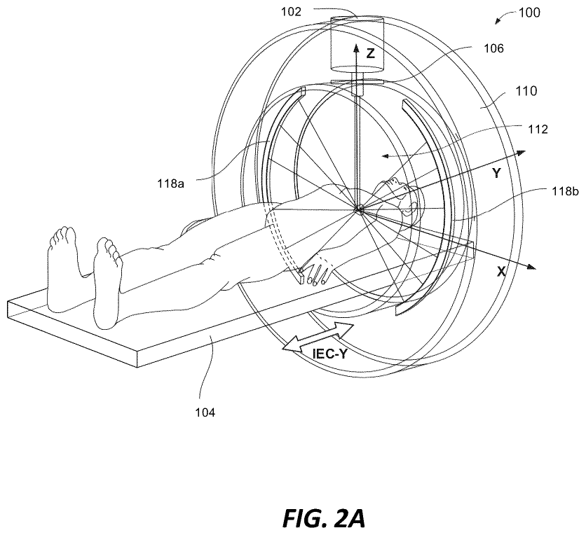

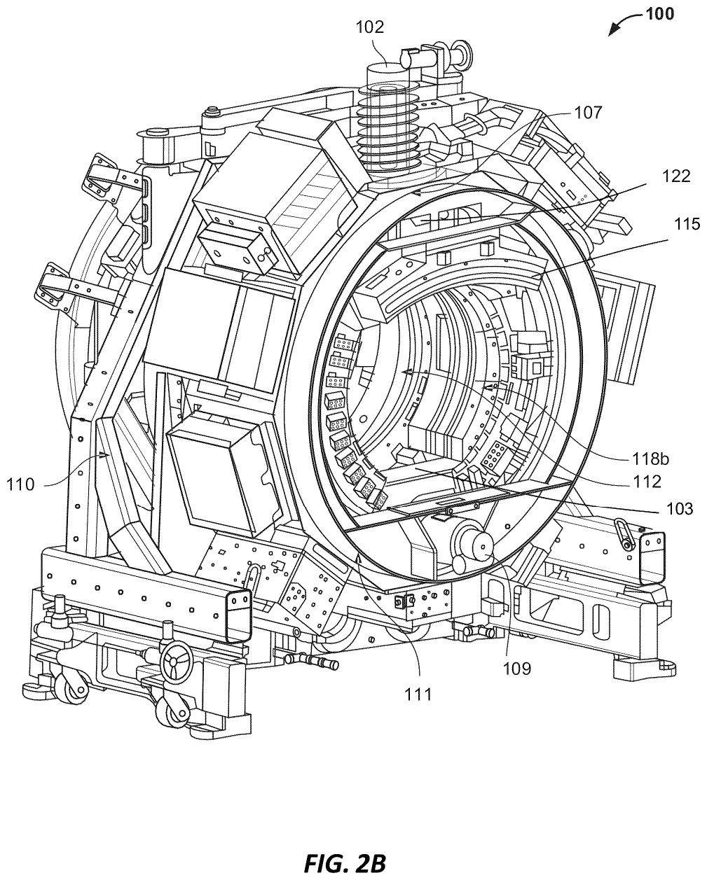

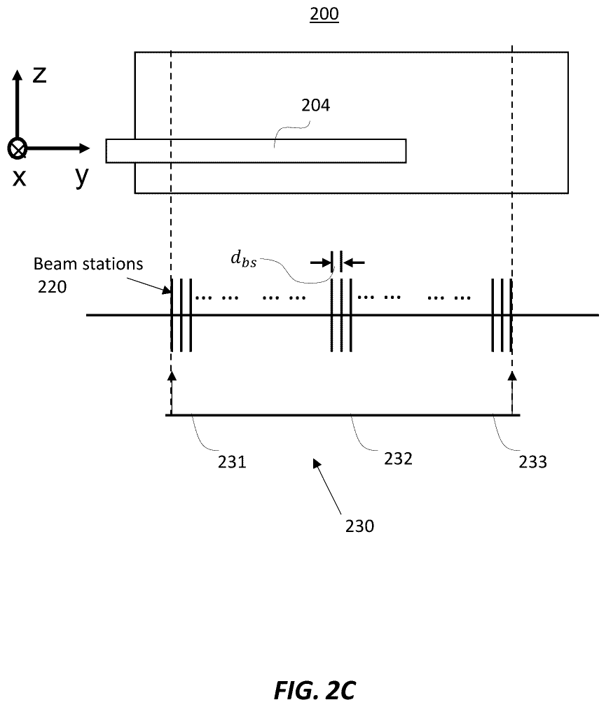

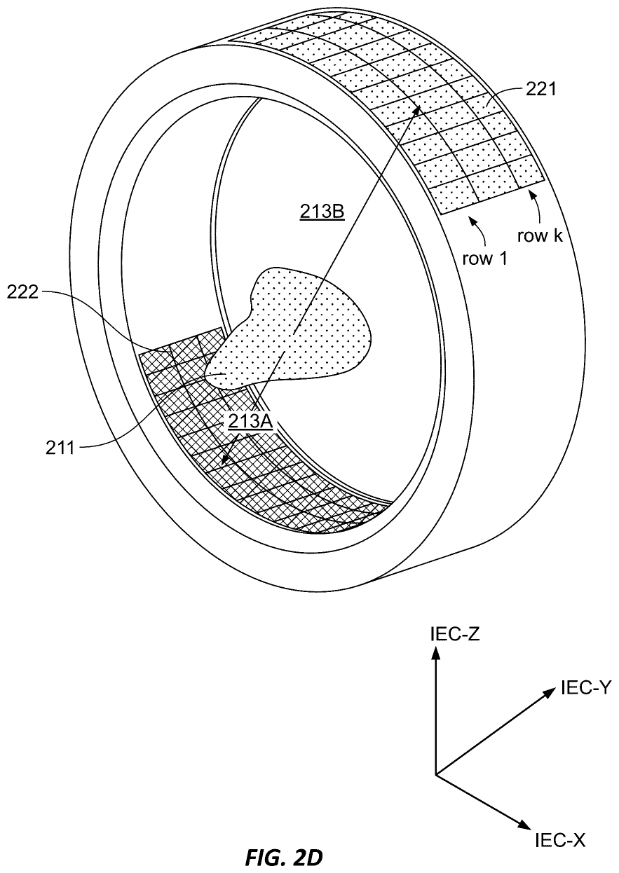

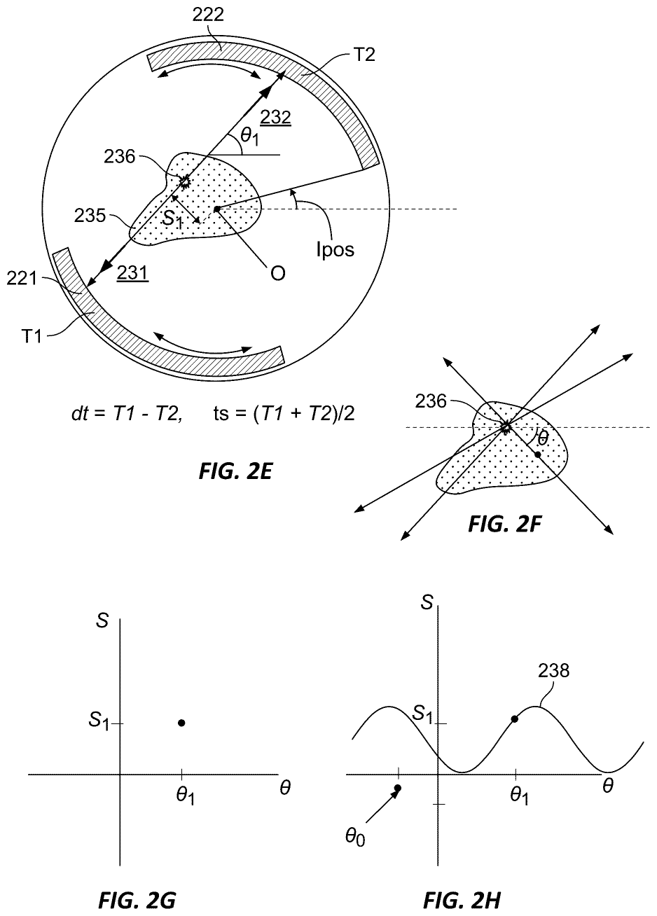

FIG. 2A depicts one variation of a radiotherapy system 100. Radiotherapy system 100 may include a gantry 110 rotatable about a patient treatment region 112, one or more PET detectors 108 mounted on the gantry, a therapeutic radiation source 102 mounted on the gantry, a beam-shaping module 106 disposed in the beam path of the therapeutic radiation source, and a patient platform 104 movable within the patient treatment region 112. In some variations, the gantry 110 may be a continuously-rotating gantry (e.g., able to rotate through 360° and/or in arcs with an angular spread of less than about 360°). The gantry 110 may be configured to rotate from about 20 RPM to about 70 RPM about the patient treatment region 112. For example, the gantry 110 may be configured to rotate at about 60 RPM. The gantry may also be configured to rotate at a slower rate, e.g., 20 RPM or less, 10 RPM or less, 1 RPM or less. The beam-shaping module 106 may include a movable jaw and a dynamic multi-leaf collimator (MLC). The beam-shaping module may be arranged to provide variable collimation width in the longitudinal direction of 1 cm, 2 cm, or 3 cm at the system iso-center (e.g., a center of a patient treatment region). The jaw may be located between the therapeutic radiation source and the MLC or may be located below the MLC. Alternatively, the beam-shaping module may include a split jaw where a first portion of the jaw is located between the therapeutic radiation source and the MLC, and a second portion of the jaw is located below the MLC and coupled to the first portion of the jaw such that both portions move together. The therapeutic radiation source 102 may be configured to emit radiation at predetermined firing positions (e.g., firing angles 0°/360° to) 359° about the patient treatment region 112. For example, in a system with a continuously-rotatable gantry, there may be from about 50 to about 100 firing positions (e.g., 50 firing positions, 60 firing positions, 80 firing positions, 90 firing positions, 100 firing positions, etc.) at various angular positions (e.g., firing angles) along a circle circumscribed by the therapeutic radiation source as it rotates. The firing positions may be evenly distributed such that the angular displacement between each firing position is the same.

FIG. 2B is a perspective component view of the radiotherapy system 100. As shown there, the beam-shaping module may further include a primary collimator or jaw 107 disposed above the binary MLC 122. The radiotherapy system may also include an MV X-ray detector 103 located opposite the therapeutic radiation source 102. Optionally, the radiotherapy system 100 may further include a kV CT imaging system on a ring 111 that is attached to the rotatable gantry 110 such that rotating the gantry 110 also rotates the ring 111. The kV CT imaging system may include a kV X-ray source 109 and an X-ray detector 115 located across from the X-ray source 109. The therapeutic radiation source or linac 102 and the PET detectors 118a and 118b may be mounted on the same cross-sectional plane of the gantry (i.e., PET detectors are co-planar with a treatment plane defined by the linac and the beam-shaping module), while the kV CT scanner and ring may be mounted on a different cross-sectional plane (i.e., not co-planar with the treatment plane). The radiotherapy system 100 of FIGS. 2A and 2B may have a first imaging system that includes the kV CT imaging system and a second imaging system that includes the PET detectors. Optionally, a third imaging system may include the MV X-ray source and MV detector. The imaging data acquired by one or more of these imaging systems may include X-ray and/or PET imaging data, and the radiotherapy system controller may be configured to store the acquired imaging data and calculate a radiation delivery fluence using the imaging data, for example, in a BgRT session. Some variations may further include patient sensors, such as position sensors and the controller may be configured to receive location and/or motion data from the position sensor and incorporate this data with the imaging data to calculate a radiation delivery fluence. Additional descriptions of radiotherapy systems that may be used with any of the methods described herein are provided in U.S. Pat. No. 10,695,586, filed Nov. 15, 2017.

The patient platform 104 may be movable in the treatment region 112 to discrete, pre-determined locations along IEC-Y. These discrete, pre-determined locations may be referred to as “beam stations”. In one variation, different beam stations may vary only by their location along the IEC-Y axis (e.g., longitudinal axis); each beam station may be identified by its location along IEC-Y. Alternatively, or additionally, beam stations may vary by the platform pitch, yaw, and/or roll positions of the patient platform. For example, a radiotherapy treatment planning system may specify 200 beam stations, where each beam station is about 2 mm (e.g., 2.1 mm) apart along IEC-Y from its adjacent beam stations. During a treatment session, the radiotherapy treatment system may move the patient platform to each of the beam stations and may stop the platform at a beam station while radiation is delivered to the patient. In some variations, after the platform has been stepped to each of the 200 beam stations in a first direction (e.g., into the bore), the platform may be stepped to each of the 200 beam stations in a second direction opposite the first direction (e.g., out of the bore, in reverse), where radiation is delivered to the patient while the platform is stopped at a beam station. Alternatively, or additionally, after the platform has been stepped to each of the 200 beam stations in a first direction (e.g., into the bore) where radiation is delivered at each of the beam stations, the platform may be moved in reverse so that it returns to the first beam station. No radiation may be delivered while the platform is moved back to the first beam station. The platform may then be stepped, for a second time, to each of the 200 beam stations in the first direction for a second pass of radiation delivery. In some variations, the platform may be moved continuously while radiation is delivered to the patient and may not be stopped at beam stations during the delivery of therapeutic radiation. Additional descriptions of patient platforms that may be used with any of the radiotherapy systems and methods described herein are provided in U.S. Pat. No. 10,702,715, filed Nov. 15, 2017, which is hereby incorporated by reference in its entirety.

FIG. 2C shows a schematic drawing of a radiotherapy system 200 extending in an IEC-Y direction according to an example BgRT imaging system, a patient platform 204, and a set of beam stations 220. The beam stations 220 correspond to position of the patient platform 204 at which the PET images are collected and/or at which a radiotherapeutic treatment is administered. In some variations, multiple beam stations 220 are each positioned from another one by a distance d_{bs}, where d_{bs} may be less than the width of a radiation beam, e.g., a few millimeters. For example, the spacing d_{bs} between the beam stations 220 may correspond to the spacing along the IEC-Y direction of PET image slices. In some variations, the spacing between the beam stations 220 may be about the same as a spacing between image slices as obtained by a typical CT imaging system. For example, the spacing d_{bs} between the beam stations 220 may be 1-6 mm, e.g., about 1.5 mm, 2 mm, 2.25 mm, 3 mm, etc. In some variations a large number of beam stations may be used (e.g., a few tens of beam stations, about hundred beam stations or more than hundred beam stations may be used).

In some implementations, a distance between the beam stations 220 may vary. For example, the beam stations 220 may be more closely positioned to each other at a starting portion 231 of a scanning section 230 and at an ending portion 233 of the scanning section 230. Alternatively, the beam stations 220 may be more closely positioned to each other in a middle portion 232 of the scanning section 230. The distance between beam stations may be determined at least in part by the location of the target region on the patient platform, and/or the planned dose distribution. For example, platform positions that would place the patient within the treatment plane with a high dose gradient may have beam stations that are closer together, and while platform positions that would place the patient within the treatment plane with a low dose gradient (or no dose at all) may have beam stations that are further apart.

The patient platform or couch motion trajectory during the acquisition of PET imaging data in a BgRT radiotherapy system may be different from the patient platform or couch motion trajectory during the acquisition of PET imaging data in a diagnostic PET imaging system. For example, the diagnostic PET system may use five discrete bed positions to acquire the whole-body PET scan, but the BgRT radiotherapy system may use a larger number of discrete beam stations (e.g., a few tens of discreate beam stations) which are separated by a few millimeters (mm) such as 2 mm, 3 mm, 4 mm, and the like. The imaging time for diagnostic PET imaging system may be a few minutes while the imaging time for the BgRT radiotherapy imaging system may be a few tens of seconds (e.g., approximately 20 second, 30 seconds, 40 seconds, and the like) per beam station. In some variations, for example, variations with a computer-generated PET image from a virtual anatomical phantom (e.g., a noiseless PET image of an xCAT phantom), there may not be a defined couch motion trajectory. A beam station simulation may model the couch trajectory that may be during the acquisition of PET imaging data on a BgRT radiotherapy system. Alternatively, instead of discrete steps, any continuous bed trajectory may be modeled.

FIG. 2D shows an example acquisition of PET imaging data (which comprises LOR data) using PET detectors 221 and 222 located on a rotatable gantry. Herein, the detectors 221 and 222 are also respectively referred to as a first detecting arc and a second detecting arc. The detectors 221 and 221 may be arranged in detector rows or rings that may each span 90°. The example detector rows (row 1 and row k) are shown for detector 221. It should be noted that any number of detector rows can be used (e.g., a few rows, a few tens of rows, or a few hundreds of rows).

Note that it is not necessary in all instances to have a rotating gantry and in some variations, the PET detectors may form a continuous full ring. During PET image acquisition, the positrons emitted by a PET tracer annihilate with electrons resulting in two almost co-linear 511 keV gamma photons, which define a line-of-response (LOR) or emission path. FIG. 2D shows a PET-avid region 211 (e.g., a region of an anatomy that has taken up a PET tracer) that emits gamma photons 213A and 213B traveling in opposite directions towards detectors 221 and 222 that are opposite to each other. The LOR defined by the two photons 213A and 213B may be detected by the detectors 221 and 222 and LOR's detection parameters may be recorded. The detection parameters may include a time stamp of the LOR detection (e.g., an average time at which the detectors received the gamma photons 213A and 213B), the angular orientation of the LOR (e.g., an angle the LOR makes with an IEC-X axis shown in FIG. 2D, angle of the LOR after it has been shifted to the center of the gantry or PET imaging system field-of-view), a perpendicular offset distance to a center of the gantry, and/or a time difference between a first time T1 at which a first gamma ray is detected by detector 221 and a second time T2 at which a second gamma ray is detected by detector 222. Optionally, in the example of a rotatable gantry, the detection parameters of an LOR may include the rotational location or index of the gantry. Multiple LORs from the PET-avid region 211 may facilitate the determination of the location of positron annihilation events using any suitable approach (e.g., a filtered back-projection approach, time of flight (TOF) techniques, or iterative reconstruction techniques). FIG. 2D shows an example IEC coordinate system that includes IEC-Y axis directed along a longitudinal axis of a bore of the PET imaging system or BgRT radiotherapy system, and IEC-X and IEC-Z axes directed perpendicular to IEC-Y axis.

An example LOR 235 comprising gamma rays 231 and 232 may originate from a single positron annihilation event in a volume of tissue 236 (e.g., a voxel of tissue) is shown in FIG. 2E. The LOR can be characterized or defined by an angle θ₁ and a distance S₁ drawn normal to the LOR 235 from an origin O (e.g., center) of the gantry. In one variation, detector 221 receives the gamma ray 231 at time T1, and detector 222 receives the gamma ray 232 at time T2, with a difference in detection time of dt=T1−T2. In some variations, the time window dt is chosen such that the signals received by detectors 221 and 222 correspond to a particular LOR (e.g., the dt is sufficiently small such that there is a high probability of detected signals corresponding to the same LOR). The signals received outside time window dt are not considered to correspond to that particular LOR. Some PET scanners may also use a “weighting” of the coincidence detection depending on the time difference of the two photons. For each emitted pair of gamma rays 231 and 232 corresponding to LOR 235, a time stamp ts is recorded. The time stamp may be given as an average time (T₁+T₂)/2 and indicates the detected time of LOR 235.

Some PET imaging systems, such as time-of-flight (TOF) PET imaging systems, use a precise measurement of dt=T1−T2 combined with accurate knowledge of the detector geometry to calculate the location of the emission event (herein also referred to as the annihilation event) in a physical space. For such PET imaging systems, no elaborate reconstruction techniques may be required to create an image.

In some variations, the detectors 221 and 222 of the rotating gantry are configured to rotate about a center of the gantry at a target rotational rate, further, the simulated LORs are detected for each time interval corresponding to angular position of the detectors 221 and 222. For a rotating gantry, the locations of detectors 221 and 222 are characterized by lpos gantry angle, as shown in FIG. 2E (detectors 221 and 222 are mounted on the gantry and move together with the gantry). In some variations, if a simulated LOR associated with an emission event is not “detectable” by detectors 221 and 222 due to the position of the gantry at the time the emission event (i.e., the LOR does not intersect any of the detectors 221, 222), this simulated LOR may be discarded.

In some variations, LOR data may be represented graphically by a sinogram. The position of an LOR may be characterized by a detection angle θ and an offset distance S (e.g., angle θ₁ and S₁ are shown in FIG. 2E). A sinogram is a plot that depicts the positional information of one or more LORs, where the detection angle of the LOR(s) may be along one axis and the offset of the LOR(s) from the center of the field-of-view may be along the other axis. Thus, a sinogram can be represented as {θᵢ, Sᵢ} for a given LORᵢ. One axis of the sinogram (e.g., horizontal axis) may be the LOR detection angle θ (i.e., angle ranging between 0 and 360 degrees measured from a line parallel to one of the central axes of the PET detector arrays). In some variations, angle θ may include discrete values of specific angular increments measured from a line parallel to one of the central axes). The other axis of the sinogram (e.g., vertical axis) may be an offset of the LOR from the center of the PET imaging system (measured perpendicular from the LOR to the central axis). A point on a sinogram may represent an LOR event (also referred to as an LOR count) that was detected by PET detectors located at a particular angle, with a particular offset value from the center of the PET imaging system field-of-view.

The system parameters may help specify the location of the PET detector arrays of a BgRT radiotherapy system during PET signal acquisition, as well as the acquisition time available at a beam station. For example, the beam station location may define a location along the longitudinal axis (i.e., IEC-Y axis), the dwell time may define the amount of time available for the PET detectors to acquire the PET signal at that location (i.e., longer acquisition time results in more detected LORs), and the data about the number and rate of rotation may define the location of the PET detectors at the time an LOR is detected.

In some variations, the same volume or voxel of tissue 236 may emit multiple positrons that result in multiple LORs, as depicted in FIG. 2F. FIG. 2G depicts one LOR having a detection angle of θ₁ and an offset value of S₁. Each of multiple LORs from the voxel 236 may have different detection angles θ and offsets, and may each be represented as a different point in a coordinate system θ, S, as shown in FIG. 2H, forming a sinogram. As depicted there, the sinogram includes multiple LORs from the same voxel and may be represented as a sine plot 238. Thereby, a single sine plot 238 may represent a set of LORs that are emitted from a single voxel. LORs from different voxels may be represented by multiple sine plot (similar to sine plot 238), thereby forming a combined sinogram (or just simply a sinogram) for the annihilation emission data.

Alternatively, or additionally, LOR data may be recorded as a list of LORs (i.e., LOR list mode data) with each i^{th} LORᵢ having a recorded angle θᵢ, a recorded distance Sᵢ to the origin, a time stamp ts, and a time difference dt. Additional parameters may be also stored as a part of the list of LORs based, for example, on particular configuration and type of PET imaging system. For example, coordinates of detectors for detecting an LOR may be stored together with a position (e.g., angular position lpos) of a rotating gantry as well as time tpos at location lpos.

It should also be noted that when LORs are detected which traverse the Y-axis, they are frequently “re-binned” into LORs that all correspond to single planes (slices) that usually represent the multiple rows of detectors (say along the y axis).

As discussed above, the collected LOR data may be represented by a sinogram or by list mode data as described above. When the LOR data is represented by a sinogram, the sinogram may be divided into sections (sinogram bins Bin(i, j,k)) with each bin containing a range of angles Θᵢ=θᵢ±dθ, and range of normal distances Sⱼ=sⱼ±ds and a given re-binning plane (for example, a detector row) k. Thus, LORs in a particular sinogram bin Bin(i, j, k) may have about the same angles θᵢ and about the same normal distances s; and the same re-binning plane k. Therefore, Bin(i, j, k) corresponds essentially to LOR(i,j) in plane k.

### Methods

Since BgRT uses real-time emissions from a PET tracer (e.g., a PET signal, lines-of-response or LORs) to guide the delivery of radiation to a target region, some methods of BgRT planning include a patient-specific PET signal evaluation. The PET signal evaluation may help determine whether the PET signal has characteristics that are suitable for guiding radiation delivery. The suitable characteristics may be defined in terms of one or more of activity concentration, PET imaging contrast between the target region and surrounding areas, and/or a dose calculation based on the PET signal itself. In some variations, the patient-specific PET signal evaluation may be conducted using the PET image that was used to diagnose and/or characterize the disease state of the patient. Typically, such diagnostic PET images are acquired on PET imaging systems with a full ring of PET detectors, with long image acquisition times, and thus, are generally considered to be of high quality (e.g., having good a signal-to-noise ratio, little noise or relatively noiseless, high contrast between the target region and surrounding tissue). Such PET imaging systems are herein referred to as diagnostic PET imaging systems. However, some radiotherapy systems used for BgRT (such as any of the examples provided herein) may not have a full ring of PET detectors. Instead, such PET imaging systems may have detectors that are arranged in arcs, e.g., two partial rings. As a result, there may be a reduced quantity of PET signal acquired as compared to a diagnostic PET imaging system with a full ring of PET detectors. Moreover, there may be signal artifacts that appear on PET imaging data acquired by a PET imaging system that is onboard a radiotherapy system that are not present on a diagnostic PET imaging system. Therefore, the characteristics of the diagnostic image may not reflect the actual quality of the PET imaging data or PET signal acquired on the PET imaging system of a BgRT radiotherapy system (herein referred to as a BgRT PET imaging system). The BgRT PET imaging systems may have a relatively short PET signal acquisition time, reduced detector efficiency, narrower or smaller field-of-view, or other features, parameters, and the like that may introduce noise and/or imaging artifacts that are not present in diagnostic PET imaging systems. The PET images taken using BgRT PET imaging systems may be used in the BgRT delivery process, during treatment planning and subsequently during radiation delivery. While the examples provided herein are described in the context of a PET imaging system onboard a radiotherapy system (e.g., BgRT PET imaging systems), it should be understood that other PET imaging systems not related to BgRT radiotherapy systems may also be of lower quality (e.g., having a lower signal-to-noise ratio) than diagnostic PET imaging systems.

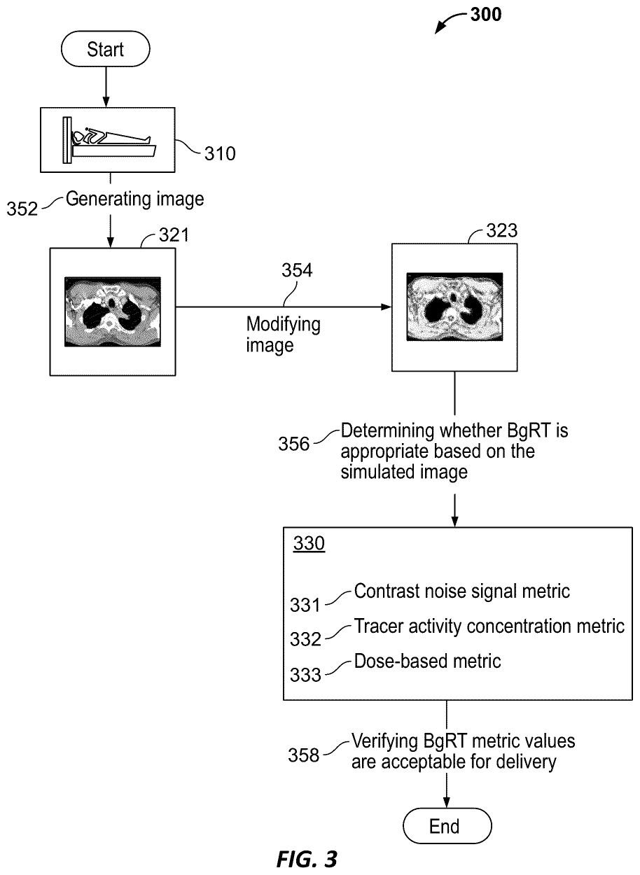

To determine whether BgRT is suitable for a patient, PET imaging data similar to the PET imaging data acquired during the BgRT therapy session may be analyzed as further described by a method 300, as shown in FIG. 3. The method 300 may include generating 352 a diagnostic PET image 321 from PET imaging data acquired using a diagnostic PET imaging system 310, and modifying 354 the PET image 321 to obtain simulated imaging data and generating a simulated PET image 323. The simulated PET image 323 may include image artifacts and noise that are consistent with PET images obtained using the BgRT PET imaging system. For example, the simulated image 323 may include imaging artifacts that may be specific to the PET detectors of a BgRT radiotherapy system, their relative arrangement (e.g., as pairs of opposing PET detector arcs instead of a full ring of PET detectors), scatter (e.g., from the linac), and the manner in which the PET imaging data is acquired (e.g., via patient platform beam stations instead of continuous platform motion). In some variations, simulated image 323 may be noisier and/or have additional imaging artifacts that are absent in the diagnostic PET image 321. Method 300 may further include determining 356, using a data analysis module 330 of a controller or processor of a treatment planning system and/or BgRT radiotherapy system controller, whether BgRT is appropriate based on simulated image 323. Consistent with the disclosed variations, data analysis module 330 may be configured to evaluate a contrast noise ratio (CNR) metric 331 (which may also be referred to as a normalized tumor signal or NTS), a tracer activity concentration metric 332, and a dose-based metric 333, as further described below, to determine the suitability of BgRT for treating a patient. In some variations, determining whether BgRT is suitable includes comparing each of the metrics with a range of acceptable values and/or an acceptable threshold value, and if one or more of the metrics are within the range of acceptable values and/or exceed the acceptable threshold value, then the controller may generate a notification indicating that BgRT may be appropriate. The notification may optionally include a graphical representation that includes the range of acceptable BgRT metric values and the BgRT metric values calculated from the simulated image 323. If BgRT is determined to be suitable by data analysis module 330, the method 300 may further include verifying 358 prior to administering BgRT during a treatment session, that the BgRT metric values of the PET images obtained by BgRT radiotherapy system 100 still meet the acceptable thresholds and/or are within an acceptable range. Verifying the suitability of BgRT may occur one or more times throughout the treatment session. Multiple occurrences of verification may help ensure the safety of radiation delivery throughout the treatment session.

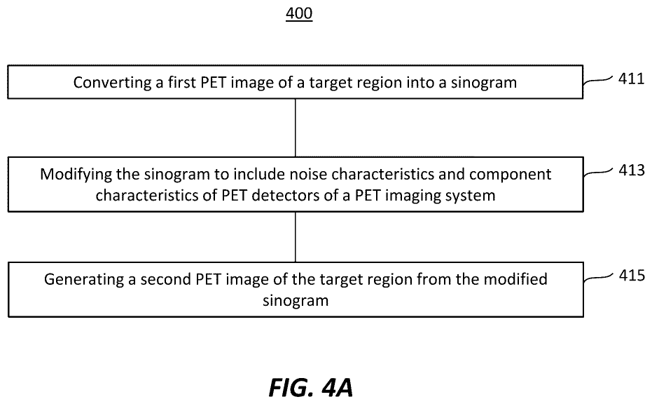

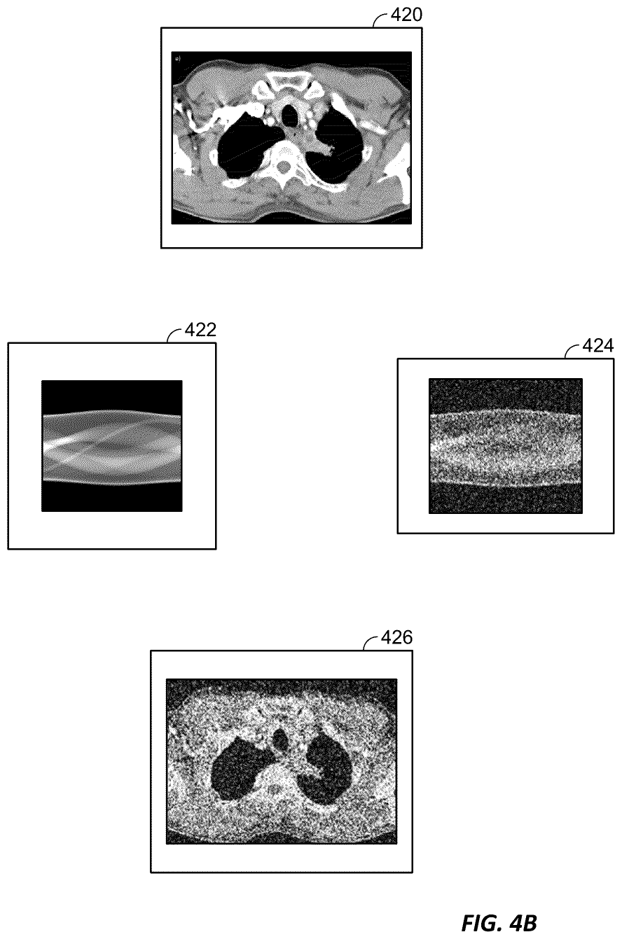

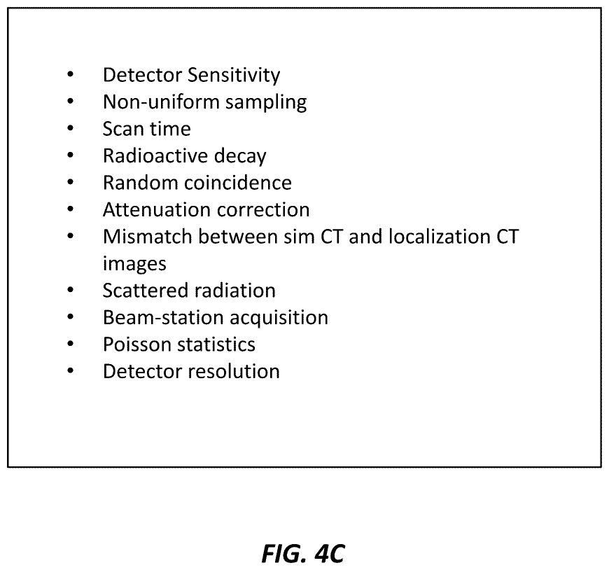

FIG. 4A depicts one variation of a method 400 of modifying a PET image to obtain simulated imaging data and generating a simulated PET image. This method may correspond to sub-steps of step 354 of the method 300. The method 400 includes converting 411 a first PET image of a target region into a sinogram. An example PET image 420 and an example sinogram 422 are shown in FIG. 4B. In one example implementation, the sinogram 422 may be generated using a PET image generated by a diagnostic PET imaging system (e.g., the diagnostic PET imaging system 310, as shown in FIG. 3).

Further, the method 400 includes modifying 413 the sinogram 422 to include imaging artifacts and noise associated with the detectors of the BgRT PET imaging system to simulate the PET signals that would be acquired using the PET detectors of a BgRT radiotherapy system. Further, the sinogram 422 may be modified by accounting for component characteristics of the detectors of the BgRT PET imaging system (e.g., by accounting that the detectors only detect a fraction of gamma rays emitted by radioactive tracer). A modified sinogram 424 is shown in FIG. 4B. The method 400 further includes generating 415 a second PET image 426 (as shown in FIG. 4B) of the target region from the modified sinogram 424. The second PET image 426 may be generated using any suitable approaches available in the art of image processing (e.g., via filtered backprojection, or iterative reconstruction techniques).

An alternative variation of the method 400 may include an imaging-only session on the BgRT radiotherapy system. The imaging-only session may comprise injecting the patient with a PET tracer (e.g., the PET tracer that will be used in the BgRT treatment session) and acquiring PET imaging data using the BgRT PET detectors on the BgRT PET imaging system, without emitting any radiation to the patient using the therapeutic radiation source. The PET imaging data acquired during the imaging-only session may be evaluated to determine whether BgRT is appropriate for a patient.

FIG. 4C further summarizes imaging system parameters and/or artifacts that may be used to modify a sinogram, as described, for example, in step 413. In one variation, the sinogram may be modified to account for the sensitivity (or sensitivities) of the second PET imaging system (e.g., the PET imaging system of a BgRT radiotherapy system) by, for example, removing some data from the sinogram (e.g., LORs) that may not be detected due to limited PET detector sensitivity and/or efficiency. Such limitations in sensitivities and/or efficiencies may arise from imperfect conversion of 511 keV photons to scintillating photons by the scintillator crystals of a PET detector, and/or sensitivity limitations of the photon detector to the scintillating photons. In some variations, the sinogram may be modified to reflect the PET detector resolution of the second PET imaging system. For example, the sinogram may be adjusted to reflect a different number of scintillator crystals and/or photon detectors of the second PET imaging system. Additionally, or alternatively, the sinogram may be modified to account for non-uniform sampling of PET signals by the second PET imaging system. For example, in the context of a PET imaging system of a BgRT radiotherapy system, there may be non-uniform sampling of PET signals due to the partial-ring PET detectors mounted on a rotating gantry (as opposed to a ring of detectors that is typically used in diagnostic PET imaging system), and/or non-uniform sampling of PET signals due to beam station acquisition (i.e., patient platform is stopped at a discrete set of locations during PET signal acquisition). In some variations, the sinogram may be modified to account for the rate of radioactive decay of a particular PET tracer (e.g., the rate of radioactive decay affects the number of positrons emitted, thus, may affect the number of LORs that are detected overall). Alternatively, or additionally, the sinogram may be modified to include non-idealities and artifacts that may arise from the detection of random photon coincidences (e.g., false coincidences may result in false LORs), attenuation and/or scatter of photons as they interact with the various tissues in a patient's body.

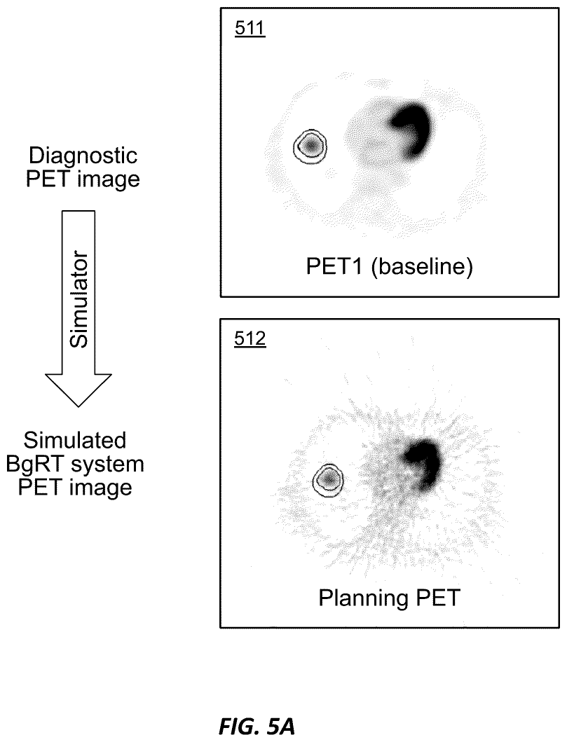

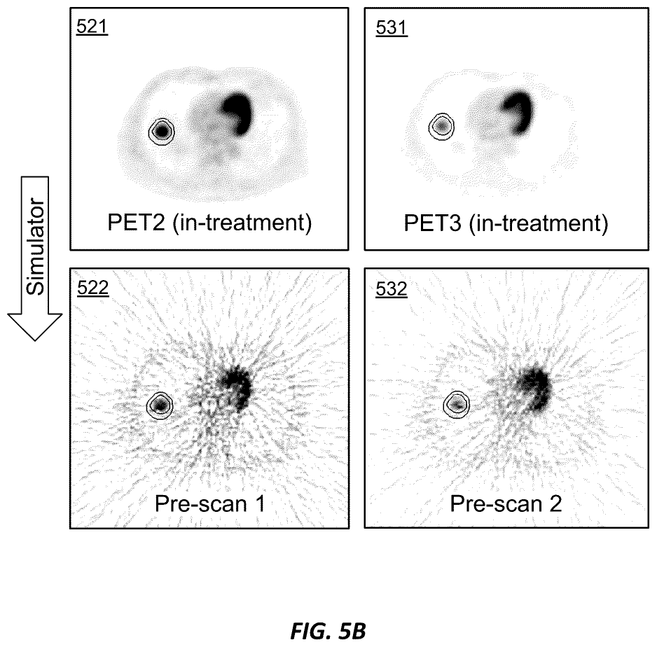

FIGS. 5A and 5B show examples of simulated BgRT radiotherapy system PET images generated from diagnostic PET images. The simulated PET images may include imaging artifacts that are present in the PET imaging systems of some BgRT systems. The imaging artifacts in the simulated PET images may be a result of one or more of the following factors: differing (e.g., reduced) PET detector sensitivity, scan times, acquisition methods, and/or resolution as compared to a diagnostic PET imaging system, Poisson statistics, non-uniform sampling, different levels of scintillator afterglow, and/or the different arrangement of the PET detectors (e.g., in two arcs vs. a full ring), and/or any of the artifacts resulting from the factors listed in FIG. 4C. In some variations, the simulated PET images may be nosier than the diagnostic PET images. FIG. 5A depicts diagnostic PET image 511 obtained using a diagnostic PET imaging system. Diagnostic PET image 511 may result in a high-resolution PET image, for example, due to diagnostic PET image 511 collected for an extended duration of time (e.g., for a few minutes or longer), and/or obtained using a highly sensitive scintillator arranged in a full ring. A simulated PET image 512 (that simulates or approximates imaging data that would be obtained by the PET detectors of a BgRT radiotherapy system) may be generated by modifying diagnostic PET image 511 using any of the methods described herein. In one variation, since a BgRT radiotherapy system is configured to collect data for a short duration of time (e.g., a fraction of a second or a few seconds) with PET detectors arranged in two opposing arcs (e.g., partial rings instead of a full ring) that may have a smaller detection area than the PET detectors used for collecting diagnostic PET image 511, PET images collected by the PET detectors of the BgRT radiotherapy system 100 may be noisier than diagnostic PET image 511. The imaging artifacts resulting from this noise may be seen in the simulated PET image 512, as shown in FIG. 5A. FIG. 5B shows several diagnostic PET images 521 and 522 that are taken for the same patient at different timepoints, with associated simulated PET images 532 and 532.

### PET Metrics

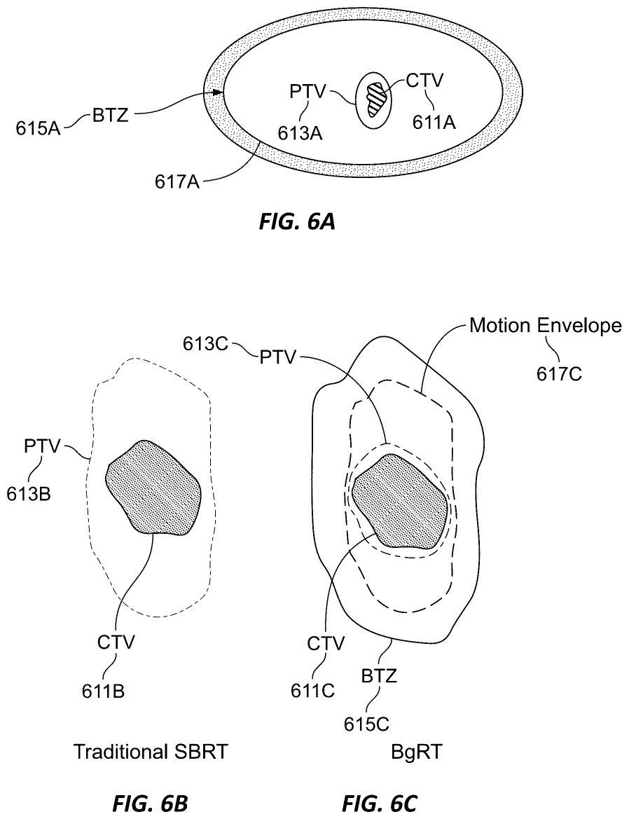

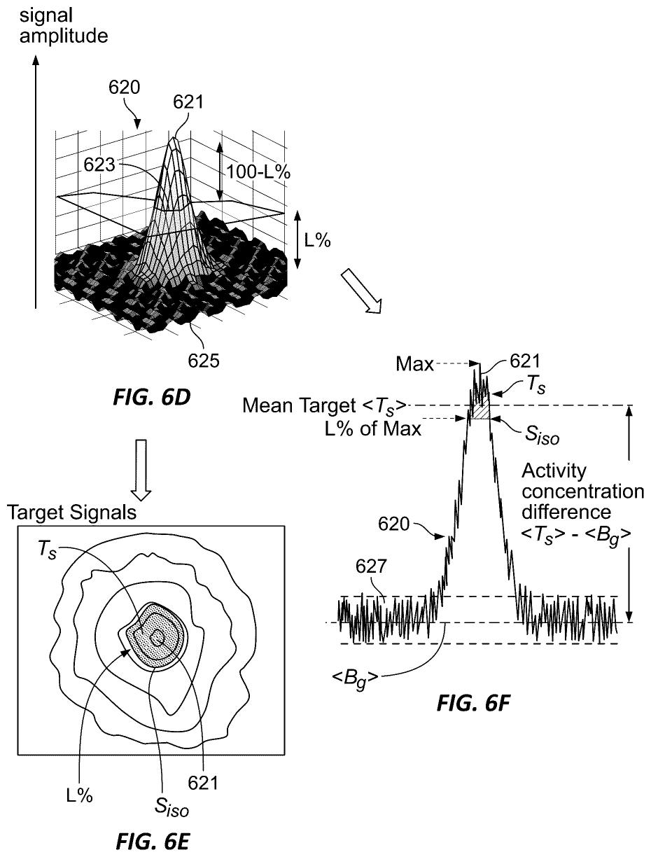

Some variations of patient-specific PET signal evaluations may include defining volumes and/or contours around the tumor and/or surrounding tissue so that the PET signals originating from the tumor may be evaluated relative to background PET signals (which may originate from surrounding tissue). FIG. 6A schematically depicts an example of a target region (e.g., a tumor), and surrounding (e.g., background) tissue. It should be understood that a three-dimensional equivalent of FIG. 6A can be used, and the example area may correspond to a volume. In some variations, the target region may be defined as a clinical target volume (CTV) region 611A, which may have a contour that encompasses the tumor. The contours of the CTV may be defined using diagnostic imaging scans and/or other methods (e.g., biopsies, presence of lymph nodes etc.) which, in the clinician's best judgement, represents the extent of the tumor and surrounding tissue that needs to be treated. CTV 611A may be surrounded by a planning target volume (PTV) 613A, as shown in FIG. 6A. PTV 613A may enclose CTV 611A with anisotropic margins to account for possible uncertainties in beam alignment, or other uncertainties (e.g., organ deformation, etc.). In some variations, the PTV defines the region that will receive the prescribed dose of therapeutic radiation.

In some variations, for example traditional stereotactic body radiation therapy (SBRT) or intensity modulated radiation therapy (IMRT) treatment, the PTV may be defined to encompass the range of motion (e.g., motion envelope) of the target to help ensure that the prescribed dose is delivered to the target even if it moves. However, unlike the PTV defined for SBRT/IMRT, the PTV for BgRT may be does not encompass the entire range of motion of the target. Because the radiation delivered in BgRT tracks the real-time location of a target, even if it moves, the entire motion path of the target and the conventional setup margin may not part of the PTV expansion. Instead, for BgRT, another volume is defined by the clinician that encompasses the motion range of the target. This volume, which may be referred to as the biology tracking zone or BTZ, may be used as a mask or filter to remove/ignore PET signals originating in other patient regions. Notably, the prescribed therapeutic dose is delivered to the PTV, but not to the entire volume of the BTZ. BTZ 615A is a volume unique to BgRT defined at the time of treatment planning which conceptually sets the boundaries within which the target is tracked. FIG. 6B shows regions CTV 611B and PTV 613B for a traditional IMRT/SBRT, and FIG. 6C shows related regions CTV 611C and PTV 613C as defined for BgRT. It should be noted that the BgRT PTV 613C is smaller than the IMRT/SBRT PTV 613B. Further, FIG. 6C shows a motion envelope 617C which is a region that contains PTV 613C at different positions due to target motion. In one variation, BTZ 615A, as shown in FIG. 6A, is surrounded by a shell region 617A. Shell region 617A can be used as a region over which a mean background signal is obtained (e.g., shell region 617A is sufficiently remote from CTV 611A, thus, it may serve as a reasonable representation of the background signal). Shell region 617A may be determined using any suitable approach. For example, a boundary of shell region 617A may include an outer boundary of BTZ 615A and have an area (or volume) that is a fraction of an area (or volume) of BTZ 615A. For instance, in one implementation boundary of shell region 617A may have an area (or volume) that is a fraction of a difference between the area (or volume) of BTZ 615A and the area (or volume) of CTV 611A. The fraction may be between 1 to 100 percent. Further, shell region 617A may be configured to be conformal to the outer boundary of BTZ 615A. Alternatively, or additionally, a shell region 617A may be a border around the BTZ that is a few pixels thick, which may represent a margin of about 1 mm to about 5 mm from the boundary of the BTZ. For example, shell region 617A may be 1-10 pixels thick with all the values and subranges in between. For example, shell region 617A may be 1 pixel thick, 2 pixels thick, 3 pixels thick, 4 pixels thick, 5 pixels thick, and the like. For example, shell region 617A may be 1 mm thick, 2 mm thick, 3 mm thick, 4 mm thick, 5 mm thick, and the like.

FIG. 6D is a conceptual depiction of a PET signal measurement 620 within a region of the BTZ. Signal measurement 620 is a plurality of signals corresponding to PET image pixels. The elevated signal values of signal measurement 620 may represent PET image pixels that may belong to a target (e.g., tumor) and the lowered signal values of signal measurement 620 may represent PET image pixels that are not part of the target, i.e., are part of a background signal. For example, signal measurement 620 includes a peak signal value 621 (which may be the pixel with the highest signal value within the BTZ), and a mean background signal 625 (herein, denoted by <Bg>). In one example, signal measurement 620 and mean background signal 625 are used to determine a CNR metric that may be used to evaluate whether the PET signal is suitable for BgRT planning and delivery. The CNR metric may be calculated using several approaches described below.

### CNR Metric

Before describing approaches for determining the CNR metric, it is instructive to introduce some definitions and describe variables used for determining the CNR metric. For example, mean background signal 625 may be calculated using several approaches. In an example implementation, mean background signal 625 may be calculated over shell region 617A. Alternatively, mean background signal 625 may be calculated over BTZ 615C, as shown in FIG. 6C, in a region that is outside CTV 611C, as shown in FIG. 6C. In one example, CTV 611C is a region that may be defined by a clinician. In one variation, CTV 611C may be defined by a contour line for which signal measurement 620 include signals that are above the lowest signal value of signal measurement 620 (as measured in BTZ 615C) by a target percentage value. For example, the boundary of CTV 611C may be defined by a contour line that has signal value 5% higher, 10% higher, 15% higher, 20% higher, 25% higher, and the like, than the lowest signal value of signal measurement 620 in the BTZ. For example, the contour line bounding CTV 611C may correspond to a signal value that is in a range of 1-80% higher than the lowest signal value of signal measurement 620. Alternatively, the contour line bounding CTV 611C may be determined by a signal value that is lower than peak value 621 by a target percentage value. For example, the boundary of CTV 611C may be defined by a contour line that has signal value 50% of the peak value 621, 60% of the peak value 621, 70% of the peak value 621, 80% of the peak value 621, 90% of the peak value 621, and the like. For example, the contour of CTV 611C may encompass the pixels within the BTZ that have a signal value that is greater than or equal to half (50%) of the peak value 621 or greater than or equal to 80% of the peak value 621. Additionally, or alternatively, CTV 611C may be defined using maximum-likelihood expectation-maximization (MLEM) method as described in a PCT Application No. PCT/US2022/017375, (“Appl. '375”) filed on Feb. 22, 2022, and which is hereby incorporated by reference in its entirety. For example, CTV 611C can be determined using CTV likelihood values as described in FIG. 5 of Appl. '375.

In some variations, calculating a CNR metric may include designating the PET image pixels that have a signal value above a target signal value as comprising the tumor. Various signal values may be represented by an isocontour values as shown in FIG. 6E. For example, an isocontour value Sᵢₛₒ may correspond to signal value 623, as shown in FIG. 6D. In one example, signal value 623 may be at a target percentage level Z of signal measurement 620 as measured from a mean background signal 625 or as measured from peak signal value 621, as shown in FIG. 6D (and FIG. 6E). For example, the target percentage level L may be 50%, 60%, 70%, 80%, or 90% of the peak value 621 and/or may be 5% higher, 10% higher, 15% higher, 20% higher, 25% higher than the mean background signal 625. Further, signals above a target signal value, herein referred to as target signals (Tₛ), are used for determining the CNR metric. For example, Tₛ may be the signal values for the PET image pixels that have signal values that are greater than or equal to Sᵢₛₒ. In some variations, Tₛ may be the average of the signal values (e.g., mean activity concentration) for the PET image pixels that have signal values that are greater than or equal to Sᵢₛₒ. FIG. 6F depicts an example of a cross-section of signal measurement 620, where the signals of pixels having signal values greater than or equal to Sᵢₛₒ are indicated as target signals Tₛ. FIG. 6F also depicts the iso-signal level Sᵢₛₒ, and fluctuations in a background 627 of signal measurement 620. Additionally, FIG. 6F shows a mean target signals <Tₛ> which is a mean value of target signals Tₛ, as well as a difference <Tₛ>−<Bg> which is defined as an activity concentration difference.

In some variations, determining CNR metric may include calculating the mean background signal 625 and a variance of the background signal (herein, denoted as σ_{Bg}). The variance of the background signal (Bg) indicates how much the background signal may be expected to deviate from mean background signal 625 (e.g., σ_{Bg}=E [(Bg−<Bg>)²], with E—being an expectation operator).

While CNR metric can be defined in a few possible ways, further discussed herein, various definitions of CNR metric may result in a CNR metric having high values when signal measurement 620 is well above the mean background signal 625, and in a CNR metric having lower values when signal measurement 620 is closer to the mean background signal 625.

In one variation, the CNR metric is computed as CNR=(<Tₛ>−<Bg>)/σ_{Bg}. Here, in the expression for CNR, Tₛ are target signals that are higher than Sᵢₛₒ. Sᵢₛₒ may be 5%, 10%, 15%, 20%, 25%, 30%, 35%, 40%, 45%, 50%, 55%, 60%, 65%, 70%, 75%, 80% of a maximum signal value detected in BTZ 615C (e.g., peak value 621), as shown in FIG. 6C. Alternatively, Sᵢₛₒ may be 5%, 10%, 15%, 20%, 25%, 30%, 35%, 40%, 45%, 50%, 55%, 60%, 65%, 70%, 75%, 80%, or the like, higher than a background value Bg. Further, as defined above, σ_{Bg} is a variance in a background signal. A CNR metric value larger than a threshold may indicate that a tumor is resolved with a sufficient contrast, CNR≥ Threshold (CNR). In one variation, Threshold (CNR) may be larger than 1, 1.5, 2, etc., e.g., 1.7, 2.1, 2.5, 2.7, 3.1. Having Threshold (CNR)>1 indicates that than difference <Tₛ>−<Bg> is larger than a background variance. In some variations, the Threshold (CNR) may have one value at the time of treatment planning (e.g., 2.7) and a different value at the time of treatment at the PET pre-scan (e.g., 2.0), which may help account for variations in the PET signal (e.g., by about +/−25%). For example, the passing threshold for the CNR metric may be lower for the PET pre-scan than the passing threshold for CNR at the time of treatment planning. Alternatively, the passing threshold for the CNR metric may be higher for the PET pre-scan than the passing threshold for CNR at the time of treatment planning.

It should be noted that the CNR metric, as defined above, is only one possible way of determining the contrast of a tumor relative to a background. In another variation, the CNR metric may be defined as CNR=MedianAC[PTV]/<Bg>. Herein, MedianAC[PTV] is a median activity concentration as determined over a PTV region, and <Bg> is a mean background signal (e.g., mean background signal 625)). The location of the PTV region may be determined, for example, using a maximum-likelihood expectation-maximization (MLEM) method which iteratively shifts the location of the target region in the initial image (e.g., an image that was used during treatment planning) to an updated location using subsequently-acquired imaging data. One variation of a method may include acquiring imaging data of a patient region that includes a tumor, generating a map of pixel tumor likelihood values by calculating a tumor-likelihood value and background-likelihood value for each pixel of the imaging data, and determining a location of the tumor by shifting a tumor contour within the imaging data to a centroid location of the map of pixel tumor likelihood values within a BTZ contour, where the BTZ contour encompasses the tumor contour. Determining the location of the tumor or PTV or CTV may further include iteratively updating the map of pixel tumor likelihood values to generate a final map of pixel tumor likelihood values such that an average pixel value within the shifted tumor contour is within a previously-defined threshold of an average pixel value within a pre-shifted tumor contour, calculating a centroid location of the final map of pixel tumor likelihood values, and determining the location of the tumor by shifting the tumor contour to the calculated centroid location. Additional details about these methods are included in Appl. '375. While the example CNR metric described here uses the median AC of the PTV, in other examples, the CNR metric may use the median AC of the CTV.

FIG. 6F shows mean target signal <Tₛ> computed as an average value of the pixels having signal values above a particular signal level (e.g., above a signal level corresponding to L % of a maximum value 621). Further, FIG. 6F shows a value of a mean background signal <Bg> and an active concentration difference between the mean target and the mean background <Tₛ>−<Bg>.

It should be appreciated that other metrics for CNR may also be used. For example, CNR metric may be defined as SNR=Iₘₐₓ (BTZ)/Iₘₑₐₙ (BTZ), where Iₘₐₓ (BTZ) is a maximum signal value (e.g., peak value 621, as shown in FIG. 6D) in a BTZ (e.g., BTZ 615C, as shown in FIG. 6C), and Iₘₑₐₙ (BTZ) is an average signal as averaged in BTZ 615C.

In some variations, to determine a CTV region and/or PTV region a computed tomography (CT) imaging data may be used in addition to PET imaging data. For example, CT imaging data facilitate the determination of a region containing a tumor, which may be determined to coincide with CTV or PTV region.

### Activity Concentration Metric

The activity concentration (AC) metric indicates a PET tracer activity concentration based on PET imaging data (which may be simulated PET imaging data or PET imaging data acquired on a PET imaging system). For example, the AC metric may include the radiation activity concentration measured in kilo becquerel (kBq) per milliliter (ml) of volume (kBq/ml). For example, the radiation activity concentration of 5 kBq/ml indicates that there are 5000 nuclear decay in one second per milliliter. In some variations, the AC metric may need to be above an activity concentration threshold for a BgRT procedure to be indicated.

### Dose-Based Metric

In some variations, the dose-based metric is determined by calculating radiation doses to one or more target regions using PET imaging data (which may be simulated PET imaging data or PET imaging data acquired on a PET imaging system) and comparing the calculated radiation doses to respective allowable clinical bounds for such radiation doses.

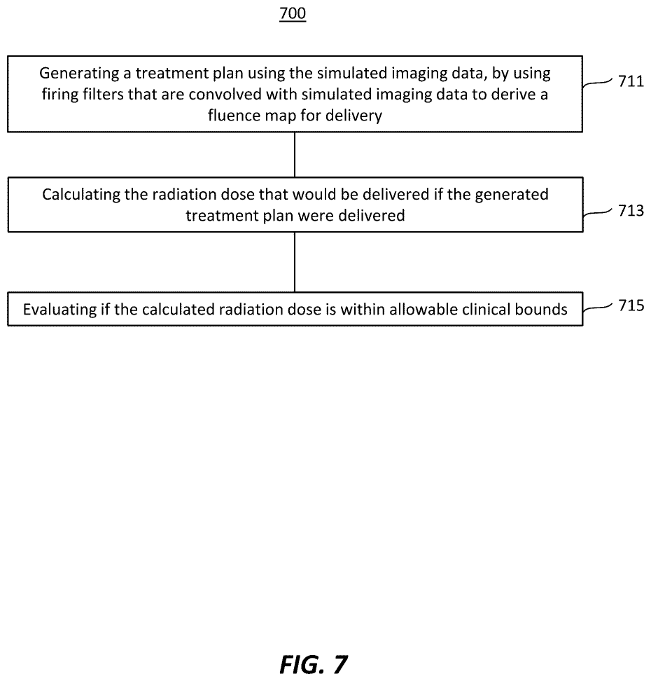

FIG. 7 shows an example of a method 700 for generating a dose-based metric. Method 700 includes generating 711 a treatment plan using the simulated imaging data, by using firing filters that are convolved with simulated imaging data to derive a fluence map for delivery. The firing filters are generated as part of planning and they are an output of BgRT planning. The inputs to treatment planning include prescribed dose(s) to the target region, dose constraints to radiation-sensitive structures (e.g., organs at risk or OARs), simulated imaging data, and CT imaging data. These inputs (as well as other constraints) are processed by an optimization algorithm to calculate firing filters that, when convolved with imaging data, generate a radiation fluence map. The fluence map may be used by the BgRT radiotherapy system 100 to deliver the prescribed dose to various tumors. Further details of calculating firing filters and using these filters for generating the fluence map are described in U.S. Pat. No. 10,688,320, filed May 30, 2018, which is hereby incorporated by reference in its entirety.

Further, method 700 includes calculating 713 the radiation dose that would be delivered if the generated treatment plan were delivered. The radiation dose may be calculated using the fluence map and a dose calculation matrix (i.e., a mapping between radiation beamlets of a fluence map and the radiation dose to each region or voxel of a target and/or patient body). The dose calculation matrix may be generated based on anatomical image data, such as tissue density data calculated from a CT image. In one variation, the radiation dose to each structure of interest (e.g., target(s), OAR(s)) may be represented by a dose volume histogram (DVH).

Further, method 700 includes evaluating 715 if the calculated radiation dose is within allowable clinical bounds. The allowable clinical bounds for radiation dose are established based on clinical historical data. If the calculated radiation dose is within allowable clinical bounds, the treatment plan may be accepted, indicating that dose-based metric is acceptable (i.e., the determined radiation doses are within allowable clinical bounds).

### Evaluating the Suitability of BgRT

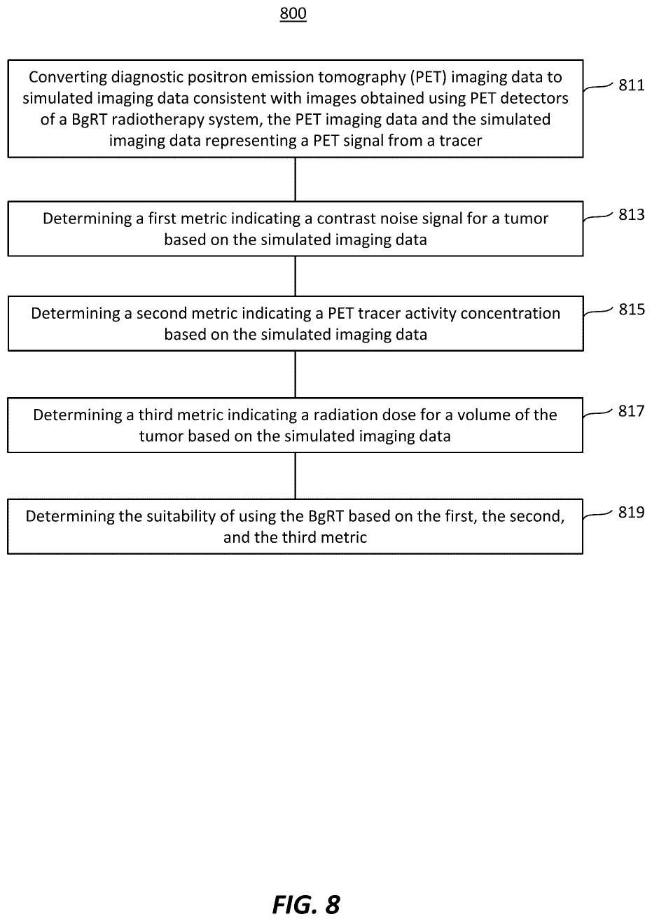

FIG. 8 is a flowchart depiction of a method 800 for evaluating the suitability of BgRT. In this example, steps of the method 800 may be performed by a suitable computing system configured for data processing. The computing system need not be operationally or physically related or connected to radiotherapy system 100, however, it may be (e.g., controller 110 may perform steps of processor 800).

The method 800 includes converting 811 diagnostic positron emission tomography (PET) imaging data to simulated imaging data consistent with images obtained using PET detectors of a BgRT radiotherapy system 100. In one variation, the computing system may be configured to convert diagnostic PET imaging data to simulated imaging data, which represent a PET signal from a tracer.

Further, the method 800 includes determining 813 a first metric indicating a contrast noise signal (CNR) for a tumor based on the simulated imaging data. The CNR metric may also be referred to as a normalized tumor signal (NTS) and may represent a relative measure between the PET signals over the tumor pixels in the simulated imaging data and the PET signals over the pixels of a background region (e.g., a shell) surrounding the tumor. In some variations, the CNR metric may be a ratio between a signal contrast value (i.e., of the average signal from the tumor) and the signal of the background region. The CNR metric may be calculated as described above.

The method 800 may include determining 815 a second metric (activity concentration metric) indicating a PET tracer activity concentration based on the simulated imaging data. In one variation, the BgRT is determined to be suitable at least based on the second metric if the second metric (e.g., activity concentration) is above an activity concentration threshold. In one example, the activity concentration threshold may be 1 to 10 kBq/ml with all the values and subranges in between. For example, the activity concentration threshold may be about 5 kBq/ml.

The method 800 may further include determining 817 a third metric (dose-based metric) indicating a radiation dose for a volume of the tumor (or target) based on the simulated imaging data. A clinician may determine that the radiation dose is clinically acceptable based on acceptable radiation dose thresholds as determined for various tissues. Alternatively, the radiation dose may be determined to be clinically acceptable by a suitable computing system configured to compare the radiation dose to the acceptable radiation dose thresholds. In some variations, the radiation dose may be calculated by converting or mapping the simulated imaging data to a radiation fluence or dose. For example, the conversion of imaging data to a radiation fluence or dose may include convolving the imaging data with a transformation matrix or firing filter to obtain a radiation fluence, and then combining the radiation fluence with anatomical images (e.g., a CT image) to obtain a radiation dose to the target region.

Further, the method 800 includes determining 819 the suitability of using the BgRT based on the first, the second, and the third metric. For example, the computing system may be configured to use all three metrics discussed above determine the suitability of BgRT. If at least one of the three metrics indicate that BgRT is not suitable (e.g., if the CNR metric does not indicate that region of a tumor has a sufficient contrast, or/and if activity concentration is below a target threshold, or/and if the radiation dose is not clinically acceptable), the BgRT is not conducted. Alternatively, if all three metrics indicate that the BgRT is suitable, the BgRT may then be allowed as a possible treatment. It should be noted that further tests may be used to determine applicability of the BgRT on a day when the BgRT is administered, prior to administering the BgRT, as further discussed below. Details of calculating the AC metric, dose-based metric, and CNR metric have been described above. Further details of how the dose-based metric, as well as examples of thresholds for these metrics are described further below.

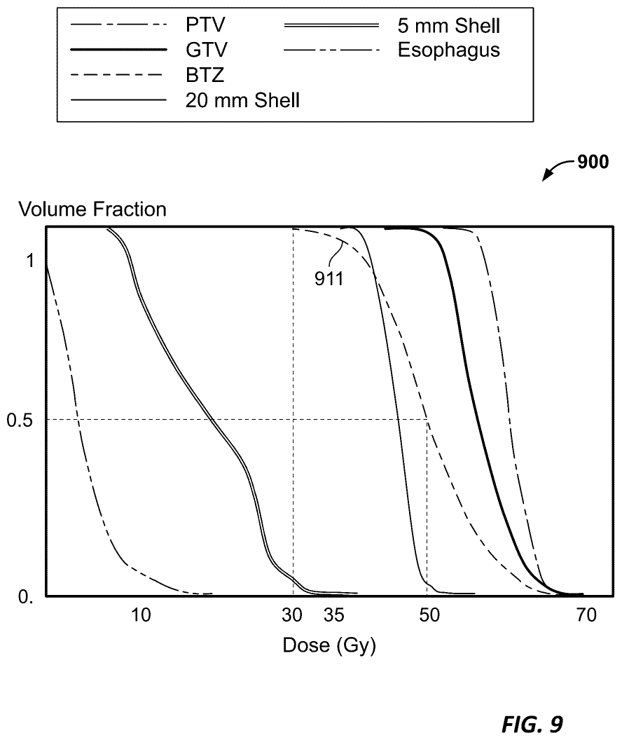

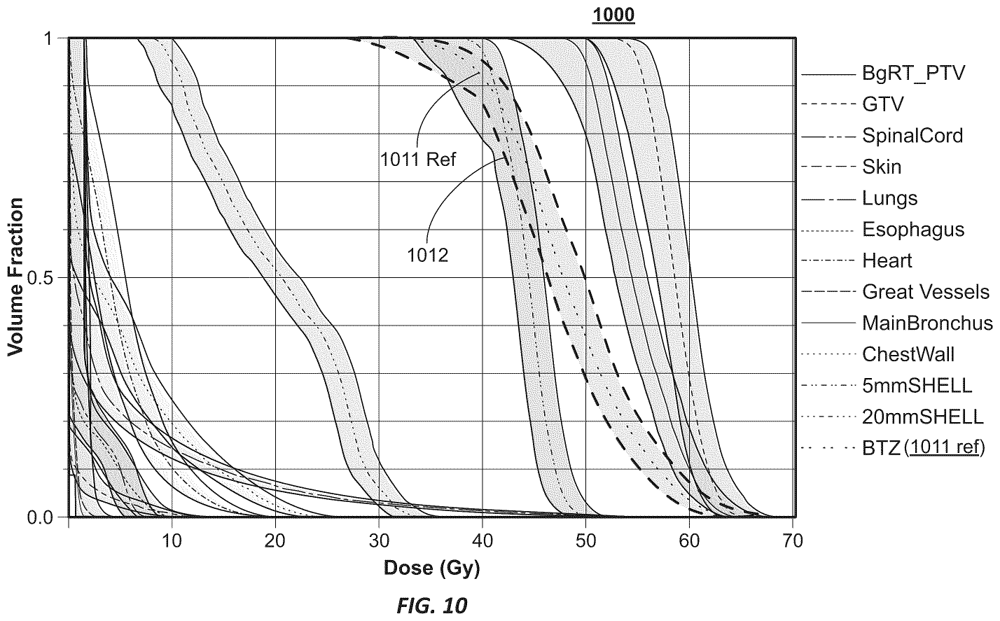

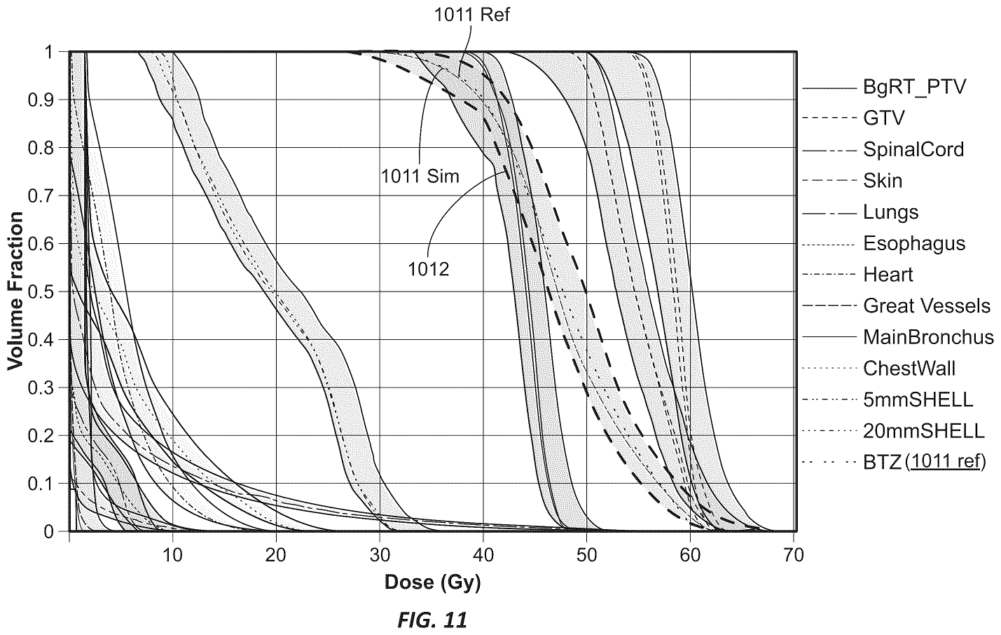

FIG. 9 depicts a plot 900 of the DVH curves for multiple structures or tissues. The DVH plot 900 represents radiation doses that may be delivered to a particular volume fraction of a tissue. The radiation doses may be calculated based on PET imaging data and/or simulations that use beam and radiotherapy machine models to calculate the dose to a volume of interest. For example, according to the DVH plot 900, about half (50%) the volume of a BTZ has or will receive about 50 Gy of radiation (as indicated by DVH 911), while an entire (100%) volume of the BTZ is configured to receive about 30 Gy (as indicated by DVH 911). The DVH plot 900 may also include a graphical representation of the radiation dose that has been, or is planned to be, delivered to a tissue, e.g., a target or an organ-at-risk (OAR). In some variations, the dose-metric may comprise a DVH for the target region, and the DVH may be compared a bounded DVH for the target region. In one variation, the calculated DVH curves for each structure or tissue may be compared with bounded DVHs that have an upper bound and a lower bound, encompassing the dose range that is clinically acceptable. For example, FIG. 10 shows a plot 1000 of DVH curves for multiple structures, with bounds indicated by regions adjacent and surrounding the DVH curve (e.g., region 1012 is the region indicating the bounds for allowable radiation doses, which may be defined by a lower bound DVH curve and an upper bound DVH curve that represent the minimum and maximum allowable doses, respectively). In one variation, DVH curve 1011 Ref is a nominal dose distribution that may represent the dose delivered without any motion or position certainties. FIG. 11 shows a DVH curve 1011 Sim, which is determined based on simulated imaging data. In one example, DVH curve 1011 Sim is within bounds described by region 1012, as shown in FIG. 11, and, thus, radiation doses represented by graph 1011 are allowable. For example, if g1(x) represent graph 1011 Ref, with x being a dose (Gy), g2(x) represents graph 1011 Sim, bu (x) represents an upper bound for region 1012, and bl(x) represents a lower bound for region 1012, then: when g1(x)−g2(x)>0, if [g1(x)−g2(x)]/[g1(x)−bl(x)]<1, then g2(x) is an allowable radiation dose. Additionally, when g1(x)−g2(x)<0, if [g2(x)−g1(x)]/[bu (x)−g1(x)]<1, then g2(x) is an allowable radiation dose. In some variations, the above requirement may be slightly relaxed. For example, in some variations, radiation doses represented by graph 1011 Sim may be (for at least some values of radiation doses) to be outside bounds 1012, and still be considered an allowable dose. For instance, when g1(x)−g2(x)>0, if [g1(x)−g2(x)]/[g1(x)−bl(x)]<Thₗ, then g2(x) is an allowable radiation dose. Additionally, when g1(x)−g2(x)<0, if [g2(x)−g1(x)]/[bu(x)−g1(x)]<Thu, then g2(x) is an allowable radiation dose. In one variation, Thₗ and Thᵤ˜1 (herein, Thₗ is used to denote lower threshold, and Thᵤ is used to denote upper threshold). For example, Thₗ and Thᵤ may be in a range of 0.95 to 1, or in any other range in proximity of 1 (e.g., between 0.9 and 1). In other words, not every point on the simulated DVH curve 1011 Sim needs to be within the bounded DVH for the dose to be considered acceptable. In some variations, if a certain threshold proportion or percentage of points of the simulated DVH curve 1011 Sim is within the boundaries of the bounded DVH, then the dose distribution may still be considered acceptable. For example, the threshold percentage of points of the simulated DVH curve that need to be within the bounded DVH may be about 80%, about 90%, about 93%, about 95%, about 97%, about 99%, etc.

As described above, in this variation, determining the suitability of BgRT may include comparing the predicted radiation dose based on PET imaging data acquired on a BgRT radiotherapy system just before a BgRT treatment session (herein also referred to as a pre-scan image) with the bounded DVH (bDVH as shown in FIG. 11) of a BgRT treatment plan. In some variations, a bDVH pass % may be defined as a percentage of points of predicted DVH curves (e.g., graph 1011 Sim) falling within planned DVH bounds (e.g., being inside region 1012), as described above. In one variation, the system may automatically calculate the bDVH pass % and this value must be greater than a selected threshold (e.g., ≥95%) to proceed with BgRT delivery. It should be noted, that while DVH curves (e.g., graph 1011 Sim) may be calculated several times for determining the suitability of BgRT procedure, reference graphs such as graph 1011 Ref and related boundary region 1012 may be determined at the time of treatment planning and may be approved by a clinician prior to a treatment session. Thus, bDVH graphs (and related information, such as bDVH bounds) form a reference against which all subsequent DVHs are evaluated.

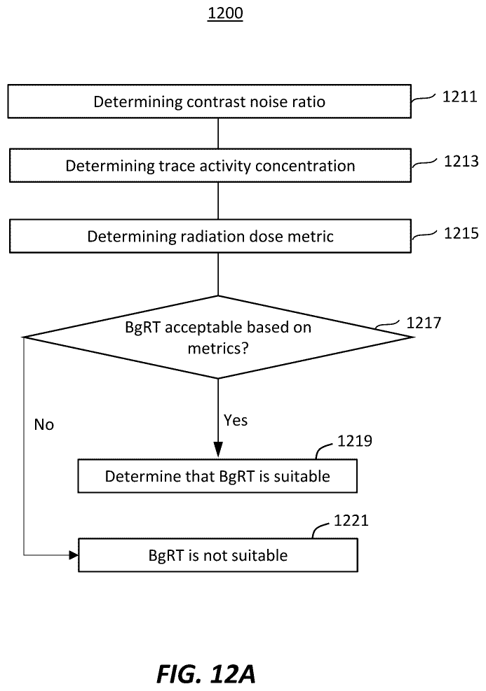

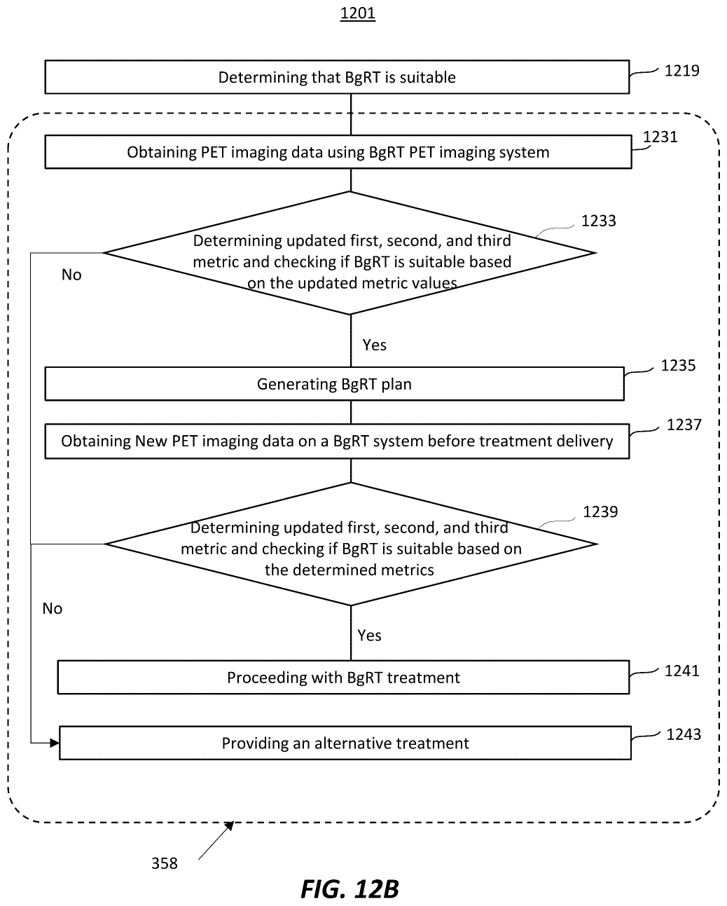

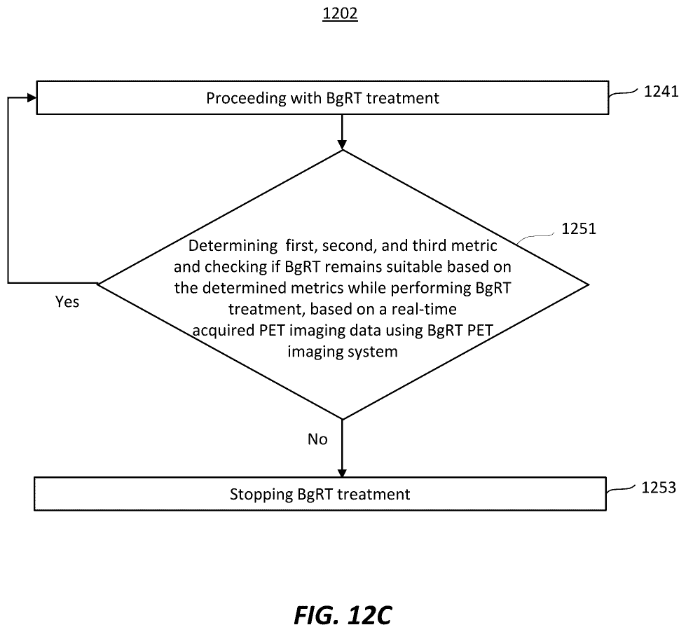

FIG. 12A shows an example method 1200 for determining the suitability of BgRT based on the three metrics as described above. In some variations, the method 1200 may be performed during treatment planning. Additionally, or alternatively, the method 1200 may be performed during a BgRT treatment session before therapeutic radiation is delivered to the patient. Further, as described above, all three metrics may be obtained based on simulated imaging data and/or pre-scan PET imaging data acquired during a BgRT treatment session before the emission of therapeutic radiation. Method 1200 may include determining 1211 the value of a contrast noise ratio (CNR) metric (first metric), determining 1213 the value of a tracer activity concentration metric (second metric), and determining 1215 the value of a radiation dose metric (third metric). The first, second and third metrics are determined using any suitable approaches described above. Method 1200 may further include evaluating 1217 whether BgRT may proceed (e.g., safe, delivers dose within clinically-acceptable ranges) based on the values of the first, the second, and the third metrics. In one variation, at step 1217, if the first metric is above a first threshold, the second metric is above the second threshold, and the third metric is above a suitable third threshold (or within acceptable bounds), it may be determined that the BgRT procedure is acceptable (step 1217, Yes). In some variations, the first threshold may be in a range of 1-3, the second threshold may be higher than 5 kBq/ml, and a third threshold may be 90-100% (e.g., 95%), e.g., such that 95% or more of the tumor volume receives a dose that is within the bounds of the bDVH. Alternatively, determining the third metric may include determining whether the radiation dose is within planned DVH bounds (or substantially within the planned DVH bounds, with only some values being slightly (e.g., by a few percent) above or below the DVH bounds). If the BgRT procedure is acceptable (step 1217, Yes), the method 1200 may include determining 1219 that BgRT treatment is suitable. Optionally, the method may comprise delivering radiation according to the BgRT treatment plan. Alternatively, if the BgRT procedure is not acceptable (step 1217, No), the method 1200 may include determining 1221 that BgRT is not suitable. The method may optionally comprise generating a notification that BgRT treatment may not be suitable.

It should be appreciated that the suitability of BgRT may be evaluated several times during treatment planning and/or delivery for a patient. For example, the suitability of the BgRT may be first evaluated when determining whether BgRT would be helpful to a patient, and evaluated again before BgRT is delivered to that patient. In some variations, during the first evaluation, the requirements (e.g., thresholds, number of BgRT metrics that pass threshold values) for determining the suitability of the BgRT procedure may be more relaxed than the requirements used for the second evaluation. For example, during the first evaluation, if one metric (e.g., the first, the second, or the third metric) is above a required associated threshold, the BgRT may be determined to be suitable. Alternatively, if at most one metric (e.g., the first, the second, or the third metric) is below the required associated threshold, the BgRT may be determined to be suitable.

Additionally, or alternatively, during the first evaluation a first set of thresholds may be used, and during the second determination, a second set of thresholds may be used. For example, during the first evaluation, the first threshold may be above 1, the second threshold may be above 2 kBq/ml, and a third threshold may be in a range of 85%-100% of points on the DVH curve that are within the bDVH. During the second evaluation narrower ranges for thresholds may be used. For example, during the second evaluation, the first threshold may be above 2, the second threshold may be above 5 kBq/ml, and a third threshold may be in a range of 95%-100% of points on the DVH curve that are within the bDVH.

FIG. 12B depicts one variation of a method 1201 which may include evaluating whether BgRT (e.g., according to the method 1200) is suitable multiple times in a BgRT workflow. In an example variation, step 1219 of the method 1201 is the same as step 1219 of the method 1200. Additionally, the method 1201 may include obtaining 1231 new PET imaging data using a PET imaging system. For example, the new imaging data may be obtained using PET imaging system of BgRT radiotherapy system 100 prior to administering a BgRT procedure. Since the new imaging data is obtained using BgRT radiotherapy system 100, it may provide a more accurate representation of the imaging data that may be acquired and used to guide radiation during the BgRT procedure.

Method 1201 may include determining 1233 updated first, second, and third metrics and checking that BgRT is suitable based on the determined metrics. For example, the suitability is determined using method 1200, while using the new first, second, and third metrics. If the suitability of BgRT is established (step 1233, Yes), the method 1201 may include generating 1235 a BgRT treatment plan. Alternatively, if the suitability of BgRT is not established (step 1233, No), the method 1201 may include providing 1243 an alternative treatment for the patient. In some variations, the alternative treatment may include IMRT or SBRT based therapy.

After a BgRT treatment plan is generated but before radiation is delivered to the patient, the values of the BgRT metrics (e.g., the first, second and third metrics of FIGS. 12A and 12B) may be re-calculated based on PET imaging data acquired just before treatment delivery, e.g., at the beginning of a treatment session. In one variation, the method 1201 may include obtaining 1237 new PET imaging data on a BgRT system before treatment delivery, and determining 1239 updated first, second, and third metrics and checking whether BgRT is still suitable based on the updated metric values. For example, the suitability may be evaluated using the method 1200 based on the updated values of the first, second, and third BgRT metrics. If the suitability of BgRT is established (step 1239, Yes), the method 1201 may include proceeding 1241 with the BgRT treatment. Alternatively, if the suitability of BgRT is not established (step 1239, No), the method 1201 may include providing 1243 an alternative treatment for the patient, such as IMRT or SBRT, or ending the treatment session.

The evaluation of whether to proceed with BgRT procedure may be repeated as many times as desired. In some variations, the frequency and/or the circumstances under which the evaluation may take place may be based on an input from a human operator. It should be noted that, in some variations, steps 1231-1243 are an implementation of step 358, of the method 300 as shown in FIG. 3.

In some variations, there may be checks throughout a BgRT treatment session to confirm that it continues to be clinically acceptable and/or safe to deliver BgRT treatment. These checks may be based on the PET imaging data acquired in the course of delivering BgRT treatment. One variation of a method of BgRT suitability checks during a treatment session is depicted in FIG. 12C. Method 1202 may include proceeding 1241 with BgRT treatment (e.g., the same as step 1241 of the method 1201), determining 1251 updated values of the BgRT metrics (e.g., calculating updated values of the first, second, and third metrics) based on PET imaging data acquired in real-time using the PET imaging system of the BgRT radiotherapy system and checking whether BgRT procedure is still safe and/or clinically acceptable in light of the updated BgRT metric values. The BgRT metrics may be calculated 1251 and updated periodically during the BgRT treatment to confirm whether to proceed with the treatment. For example, step 1251 may be performed every few tens of minutes, every few minutes, every minute, every few tens of seconds, or every second. If the BgRT treatment is determined not to be safe and/or clinically-acceptable (step 1251, No), the method 1202 may include stopping 1253 BgRT treatment. For example, if the PET tracer signal diminishes during the course of a BgRT treatment session, the diminished signal may not have sufficient contrast over the background in order for BgRT radiation delivery to continue. Alternatively, if the BgRT treatment is determined to be suitable (step 1251, Yes), treatment may continue 1241 until the prescribed dose is delivered.

The BgRT metric values may be calculated and re-evaluated multiple times during a treatment session. For example, in a treatment session where multiple target regions are to be irradiated, the first, second, and/or third metrics may be calculated using PET imaging data acquired during the session prior to the treatment of each target region to determine whether the PET signal from the target region is sufficient for BgRT delivery. That is, if there are four target regions to be irradiated during a treatment session, the first, second, and/or third PET metrics may be calculated four times (once per target region). Alternatively, or additionally, the first, second, and/or third metric values may be calculated for each target region based on the PET pre-scan data acquired at the beginning of a treatment session, where the PET signal is adjusted for each target region to account for the PET signal delay during a treatment session. For example, the PET pre-scan data, without any decay, may be used to calculate the evaluation metric values for the first target region, but for the second target region, the evaluation metric values may use the PET pre-scan data with a first amount of decay, and for the third target region, the evaluation metric values may use the PET pre-scan data with a second amount of decay, and so on. The amount of decay may be determined at least partially based on the estimated time in which the successive target regions will be irradiated, the radioactivity of the PET tracer, and any patient-specific characteristics (e.g., age, size, metabolic rate, etc.). In some variations, the passing threshold (e.g., acceptable range) for each metric may be different for each target region in order to account for the diminishing PET signal throughout the treatment session. Calculating and evaluating the first, second, and/or third PET metrics throughout a treatment session, and optionally before the treatment of each target region, may help ensure that BgRT therapy can be safely delivered to a patient. It should be noted that while all three PET evaluation metric values may be calculated for each target region, in some variations, fewer than three (e.g., one or two) of the metric values may be calculated for each target region. For example, all three PET metric values may be calculated at the start of the treatment session for the first target region, but for later PET signal evaluations and/or for the second target region onwards, one or both of the CNR (also known as NTS) metric value and the tracer activity concentration metric value may be calculated (i.e., without the dose metric) to determine whether to continue BgRT treatment.

In some variations, when determining the suitability of the BgRT procedure, the updated first, second, and third metrics, determined based on newly acquired imaging data from BgRT radiotherapy system 100, may be compared to the respective first, second, and third metrics determined based on the simulated imaging data. In some variations, if a difference between the new first and the first metric is above an allowable first difference threshold, or/and if a difference between the new second and the second metric is above an allowable second difference threshold, or/and if a difference between the new third and the third metric is above an allowable third difference threshold, it is determined that the BgRT is not suitable for a patient. In some variations, a method may comprise determining whether the values of the first, second, and third metrics are relatively consistent throughout the treatment session (i.e., variance of each of the metrics are within an acceptable range). Optionally, if the values of the first, second, and third metrics fluctuate beyond an acceptable amount, the method may comprise generating a notification to the user so that they may determine whether to pause the treatment session.

In some variations, when the BgRT with a particular PET tracer is determined not to be suitable for a patient, a different PET tracer may be used, and the determination of the suitability of the BgRT may be repeated based on the PET imaging data obtained using the different PET tracer. The threshold for each of the metrics described herein may be adjusted based at least in part on the emission and/or uptake characteristics of different PET tracers.

In some variations, simulated imaging data may also be used for generating a BgRT plan. For example, the BgRT plan may include coordinates of an identified target region (including points defining the boundary of the target region), as well as firing filters that convert PET imaging data into a radiation fluence map that results in the prescribed radiation dose being delivered to a tissue located in the identified target region.

In some variations, when determining the suitability of BgRT, the simulated imaging data may be converted into line-of-response (LOR) data, and the LOR data may be used for quality assurance purposes. For example, the LOR data may be used to test whether the firing filters in the BgRT plan result in a fluence map that delivers the prescribed radiation dose. In some variations, motion models for tissues may be used to modify LOR data to include that tissue motion, and the modified LOR data is used to evaluate the BgRT plan and determine whether the treatment plan fluence map (calculated by convolving the firing filters with the LOR data) would result in a dose distribution that is clinically acceptable in the presence of the motion approximated by the motion model.

While the examples provided herein are in the context of generating a simulated BgRT radiotherapy system PET image from a diagnostic PET image, it should be understood that these methods may also be used to generate simulated PET images from a virtual computer-generated phantom. A “noiseless” PET image may be generated by a computer for a virtual (e.g., computer-generated, digital) phantom. The simulation methods described herein may be used to generate a simulated BgRT radiotherapy system PET image, including the imaging artifacts described herein, using the noiseless PET image of the computer-generated phantom. A virtual phantom may be a three-dimensional representation (e.g., a CAD model) of a region of an anatomy of a patient. In some variations, the phantom may include a target anatomical region (e.g., a tumor) of the patient. In some variations, the virtual phantom may include physical attributes of the patient's anatomy and/or the target region (e.g., tumor(s)), including but not limited to, size, shape, and relative arrangement of anatomical structures and/or target regions, absolute and/or relative motion of one or more anatomical structures and/or target regions, and/or the tissue density of the anatomical structures and/or target regions. A virtual phantom may also include simulated PET tracer uptake kinetics and/or characteristics for the anatomical structures. One example of a computer-generated phantom is the xCAT phantom, which is a virtual anatomical model of a patient based on the “Visible Human” project. The xCAT phantom may be programmed to include the anatomical structures and target region(s) (and optionally, motion models of those anatomical structures and target region(s)) within a patient. In some variations, a model of the PET tracer uptake within each of the anatomical structures may be included as part of the xCAT phantom. A PET image generated from an xCAT phantom may be converted, using any of the methods described herein, into a simulated BgRT radiotherapy PET image. The simulated BgRT radiotherapy PET image approximates the PET signals that would be expected to be acquired if the PET detectors of a BgRT radiotherapy system were used to acquire the image of the xCAT phantom. In some variations, the noiseless PET image generated from an xCAT phantom may be converted into synthetic list mode data, which is a list that includes a series of LOR events with time stamps, where the list mode data includes the artifacts and noise present in a BgRT PET imaging system.

### BgRT Radiotherapy System Lines-of-Response (LOR) Simulator

Some methods for converting PET imaging data acquired or generated under a first set of conditions into PET imaging data acquired or generated under a second set of conditions may include generating a serialized list of synthetic LOR events (i.e., LOR counts and time stamps for each event) that simulates the LOR events acquired using the PET detectors of a BgRT radiotherapy system. The serialized list of LOR events may be referred to as list mode LOR data and comprise an ordered list of LOR events with their corresponding detection times (i.e., time stamp of when they were detected by a PET detector), and in some variations, the angle of the LOR (e.g., the angular location of the detectors that sensed the LOR) and the offset from the center of the PET field-of-view. These methods may be used to convert diagnostic PET imaging data and/or noiseless computer-generated PET imaging data of a digital phantom into list mode LOR data that includes the artifacts, noise, as well as PET detector constraints, that are present in PET imaging data acquired on a BgRT radiotherapy system.

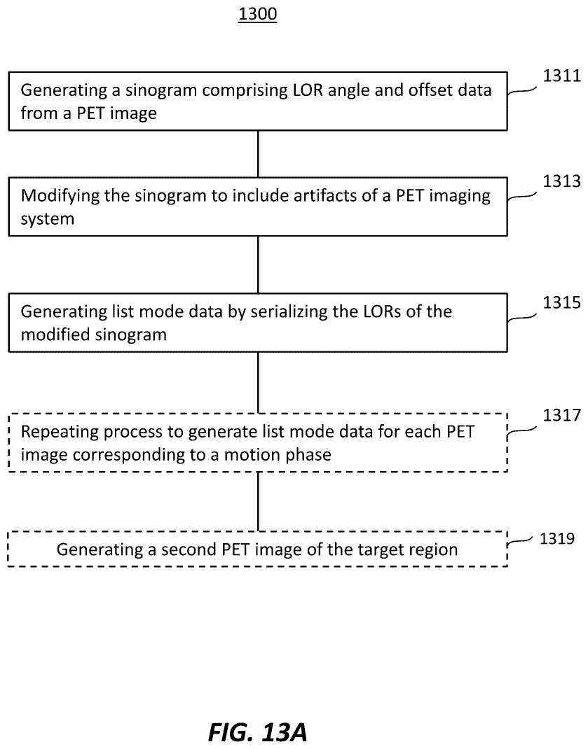

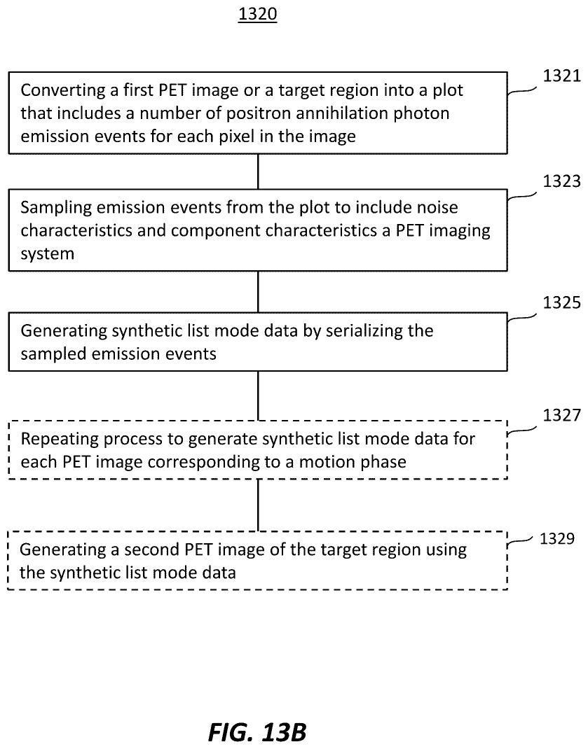

FIG. 13A shows an example method 1300 for generating simulated or synthetic list mode LOR data. Optionally, method 1300 may include generating a second PET image based on the synthetic list mode data. The synthetic list mode LOR data may simulate the list mode LOR data that may be acquired on a PET imaging system that is different from the PET imaging system that acquired the first PET image. In some variations, method 1300 may be used to generate a second PET image generated using an imaging method that is different from the imaging method used to generate the first PET image. For example, method 1300 may use a computer-generated PET image of a virtual phantom to generate synthetic list mode data and/or a PET image that simulates PET LOR data and/or a PET image acquired on an actual PET imaging system. As another example, the first PET image may be a PET image acquired on a diagnostic PET system (e.g., a PET system having a full ring of PET detectors) and the method 1300 may be used to generate synthetic list mode data and/or a PET image as if the data and images were acquired on the PET imaging system of a BgRT radiotherapy system (e.g., a PET system having partial rings or arcs of PET detectors). In some variation, method 1300 may include generating 1311 a sinogram including LOR angle and offset data from a PET image. The PET image may be obtained from a diagnostic PET imaging system or a computer-generated phantom. For example, the PET image may be obtained for an anatomy. In some variations, when anatomy is moving (e.g., lungs are moving during a breathing of a person) multiple PET images are obtained for each motion phase of the anatomy. Herein, the motion phase of the anatomy is a relatively unchanged position of the anatomy at a particular time during the motion of the anatomy. Further, the method 1300 includes modifying 1313 the sinogram to include artifacts of a PET imaging system, such as BgRT PET imaging system, as described above, for example, in relation to FIG. 4C. Additionally, the method 1300 includes generating 1315 list mode LOR data by serializing the LORs of the modified sinogram. Further details of the list mode LOR data generation are discussed below. Also, the method 1300 may optionally include repeating 1317 process of generating the list mode data for each PET image corresponding to a motion phase of the anatomy. Further, the method 1300 may optionally include generating 1319 a second PET image of the target region either by a filtered back-projection approach, a time of flight (TOF), and/or iterative reconstruction techniques.

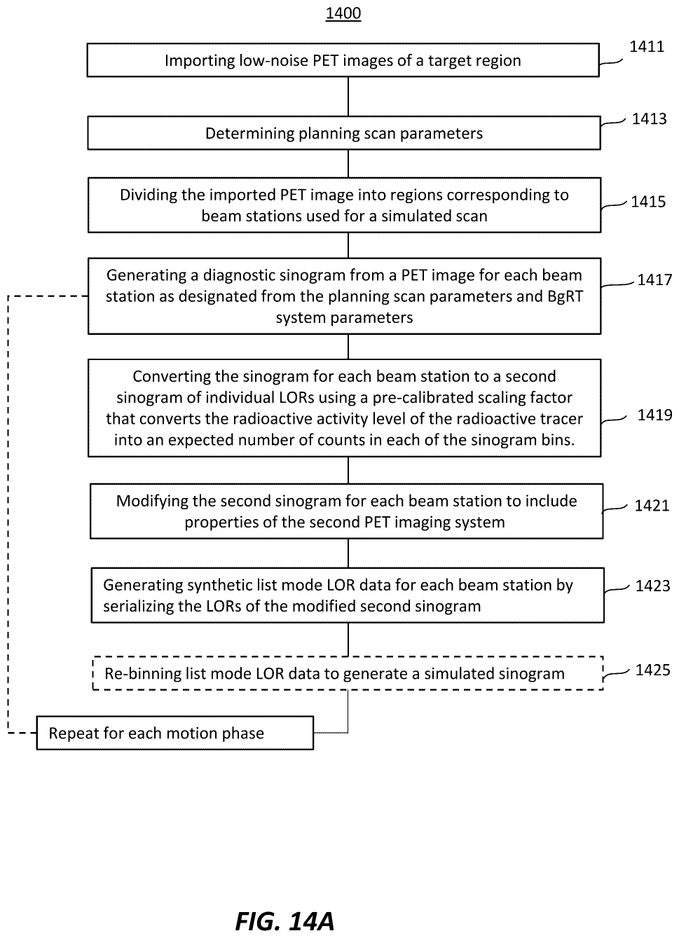

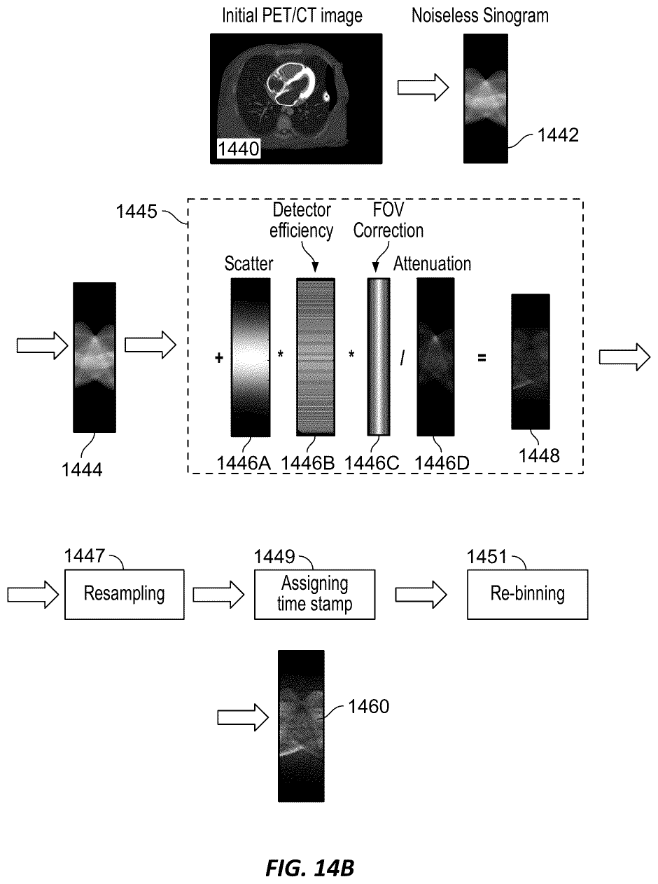

FIG. 13B is another variation of a method for generating simulated or synthetic list mode LOR data. Optionally, method 1320 may include generating a second PET image based on the synthetic list mode data. The synthetic list mode LOR data may simulate the list mode LOR data that may be acquired on a PET imaging system that is different from the PET imaging system that acquired the first PET image. In contrast with method 1300, method 1320 does not include generating a sinogram from the first PET image. As an example, method 1320 may be used to generate simulated or synthetic list mode data for PET images acquired by time-of-light (TOF) PET systems (or any PET image where each pixel or voxel of the image is represented by a number of LOR counts or emission events, e.g., the intensity at a pixel or voxel of the PET image correlates to a number of LOR counts or positron annihilation photon emission events). Method 1320 may comprise converting 1321 a first PET image of a target region into a plot that comprises a number of positron annihilation photon emission events for each pixel in a PET image, sampling 1323 emission events from the plot to include noise characteristics and component characteristics a PET imaging system, and generating 1325 synthetic list mode data from the plot by serializing the sampled emission events by assigning a time stamp to each sampled emission event. Optionally, method 1320 may comprise generating 1327 a second PET image of the target region using the list mode data, for example, by plotting an intensity level at every pixel that correlates with the number of emission events at that pixel. In some variations, the synthetic list mode data may be further modified to reflect the properties and/or characteristics of a particular PET imaging system. For example, the synthetic list mode data may be modified to account for one or more characteristics of a PET imaging system, including attenuation, and/or field-of-view, and/or detector efficiency, and/or scatter. Modifications to the synthetic list mode data may include, for example, multiplying the applying a scaling factor, selecting LORs based on the detector field-of-view, applying an efficiency multiplication factor, and/or integrating a scatter kernel for each LOR to simulate scatter throughout the image. Alternatively, or additionally, the synthetic list mode data may be converted into a synthetic sinogram, and imaging artifacts and/or corrections may be applied to the synthetic sinogram to simulate the sinogram that may result from acquiring the LOR data (e.g., PET imaging data) from a particular PET imaging system. Examples of PET imaging system properties are described further below, with reference to the sinogram modifications 1421 of method 1400 and the filters and factors depicted in FIG. 14B. The modified sinogram may be back projected to generate the second PET image. In variations where a plurality of first PET images are provided of the same region over time (e.g., 4-D PET to capture target motion), method 1320 may be repeated to generate list mode data for each PET image corresponding to a motion phase.

FIG. 14A is a flowchart representation of one variation of a method for generating list mode data (i.e., a sequence of LOR data with corresponding detection time stamps) using a diagnostic PET image or a noiseless PET image of a virtual phantom (e.g., xCAT phantom), and FIG. 14B is a conceptual depiction of the method 1400 for generating synthetic LORs that are statistically representative of the LORs (and subsequent PET images) generated by the BGRT radiotherapy system. The list mode LOR data may include the imaging artifacts that may be present in list mode data acquired on a BgRT radiotherapy system. The generated list mode LOR data consists of random events distributed in time as a Poisson process (representative of radioactive decay) modelling the random noise in the BGRT system.

The method 1400 may include importing 1411 one or more low-noise PET images and determining 1413 planning scan parameters and BgRT system parameters. Examples of low-noise PET images may include diagnostic PET images (e.g., such as PET image 1440, as shown in FIG. 14B) of a target region from a diagnostic PET imaging system or computer-generated PET images of a virtual phantom. The diagnostic PET imaging system may be a separate imaging system not associated with a BgRT radiotherapy system. Optionally, the method 1400 may comprise extracting parameters associated with the BgRT PET imaging system. These planning parameters may include number and location of beam stations, how far apart are beam stations and how many beam stations are used, location of the patient platform along IEC-Y or longitudinal axis, and/or any other parameters associated with the planning scan, such as a dwell time at the beam station, a dwell time of a gantry at a particular gantry position, a number of rotations of a gantry during which the simulated PET images are determined, a scatter observed for the BgRT PET imaging system, a number of couch passes through a therapeutic irradiation plane (i.e., the number of times the patient platform is moved through the beam stations, and the like).

The BgRT system parameters, may include parameters related to geometry of the BgRT system (e.g., such parameters may include a crystal width of the PET detector, the detection coefficients for in-plane and axial direction, location of the PET detectors, PET detector geometry, number of detectors, detection efficiency, detector crystal width, detector acquisition rate, detector resolution, detector time resolution, or any other BgRT system parameters (such as, for example, parameters associated with BgRT PET image acquisition) etc. Additional parameters BgRT PET imaging parameters may include a calibration of the BgRT PET imaging system. Herein, the calibration uses a pre-calibrated scaling factor to map the radiation intensity from an anatomy containing a radioactive tracer, which is recorded by the diagnostic PET imaging system when obtaining the diagnostic PET images, and the number of LORs that would have been recorded by a BgRT PET imaging system for that particular anatomy containing radioactive tracer. Attenuation and scatter (which may not have been included in the original phantom image) may be added to the sinogram.

The method 1400 may further include dividing 1415 the imported low noise diagnostic PET images of the target region into sets of images corresponding to beam stations that are used for the simulated scan. For example, when PET image contains multiple PET image slices, these slices can be grouped into sets of slices, each set of slices corresponding to a particular beam station of the BgRT system.

The method 1400 further includes generating 1417 a diagnostic sinogram (e.g., a noiseless sinogram 1442, as shown in FIG. 14B) from a PET image for each beam station as designated from the planning scan parameters and RT system parameters, as described above. In one variation, the diagnostic sinogram may be generated from the diagnostic PET image using forward projection (Radon Transform). Because the sinogram is generated from a diagnostic PET image (which acquires PET signals using a full ring of PET detectors and has a long acquisition time) and/or a noiseless PET image of a virtual phantom (e.g., xCAT phantom), this “idealized” or diagnostic sinogram may represent an “idealized” set of LORs that does contains little, if any, noise or imaging artifacts. In some variations, the LORs of a sinogram generated from a virtual phantom may not include or account for any of the characteristics (e.g., sensitivities of the PET detectors, location of the PET detectors, limited acquisition time, etc.) of a real-world PET imaging system.

Further, the method 1400 includes converting 1419 the diagnostic sinogram for each beam station to a second sinogram (e.g., a second sinogram 1444, as shown in FIG. 14B) of individual LORs using a pre-calibrated scaling factor that converts the radioactivity level (e.g., “intensity” of a pixel on the sinogram) of the radioactive tracer into an expected number of LOR counts in each of the sinogram bins. Converting the diagnostic sinogram into the second sinogram 1444 with individual LORs may incorporate the characteristics of the PET detectors of the second PET imaging system and/or PET tracer characteristics that may affect the number of LORs that are detectable (and therefore, the LORs that are detected). The scaling factor may represent characteristics of the PET detectors of the second PET imaging system and/or PET tracer and may be defined based on one or more of a calibration of PET detector sensitivity (e.g., the sensitivity of the PET detectors of a BgRT radiotherapy system) and/or an activity concentration of a PET tracer. A system with different PET detectors and/or geometry or that uses a different PET tracer may result in a different scaling factor. For example, for a particular PET tracer with a characteristic radioactivity concentration measured in kilo (k) becquerel per milliliter (kBq/ml), there may be an expected number of LORs or counts based on that radioactivity: LORs=scaling factor×rad.concentration. The number of LORs detectable at a beam station may also be affected by the dwell time at that beam station. The scaling factor may also incorporate the capability of the PET detectors of the second PET imaging system to detect LORs; that is, while the radioactivity of a PET tracer may result a certain number of LORs, the PET detectors may be limited in their ability to detect those LORs by their sensitivity and/or arrangement relative to where the LOR is generated, and may detect fewer LORs than were emitted by the PET tracer, and in some variations, may detect fewer LORs than were detected for a diagnostic PET image. The scaling factor may be selected to reflect these characteristics. In some variations, the scaling factor may be measured for the second PET imaging system. As an example, the scaling factor for a diagnostic PET imaging system with a full ring of PET detectors may be different from the scaling factor for the PET imaging system of a BgRT radiotherapy, which has PET detectors arranged in two opposing partial rings. When this pre-calibrated scaling factor is used to modify the relatively noise-free sinogram of a diagnostic PET image and/or noiseless PET image of a virtual phantom, the resultant (i.e., second) sinogram may have LORs that include the noise and artifacts that are present in the second PET imaging system (e.g., the PET imaging system of a BgRT radiotherapy system). In one implementation of a BgRT radiotherapy system, the scaling factor may be estimated to be from about 100 to about 5000, e.g., about 2000.

The method 1400 may further include modifying 1421 the second sinogram (the sinogram 1444, as shown in FIG. 14B) for each beam station to include properties of the second PET imaging system. Examples of such properties may include scatter, detector efficiency, attenuation etc. (e.g., a PET imaging system of a BgRT radiotherapy system), as schematically indicated by process 1445 in FIG. 14B. As shown in FIG. 14B, the second sinogram 1444 is modified to correspond to the BgRT image by using a number of factors that apply a scattering filter (scatter 1446a, as shown in FIG. 14B) and filters associated with a detector efficiency 1446b and field of view corrections 1446c. Additionally, any attenuation correction factors (>1) that were used in the diagnostic scanner may be filtered (e.g., removed as schematically indicated by a division sign in front of an attenuation 1446D). Note that in the variation with an XCAT phantom, no attenuation is assumed, and additional attenuation experienced by each LOR needs to be incorporated (e.g., added) to obtain a sinogram corresponding to the BgRT PET imaging system. The attenuation filter may be supplied with the XCAT phantom. The resulting sinogram 1448 represents a modified sinogram of the diagnostic sinogram. Note that the sinogram 1448 is still “noise free” in the sense that no random sampling has been used to obtain a sinogram that is similar to the sinogram obtained by the BgRT PET imaging system.

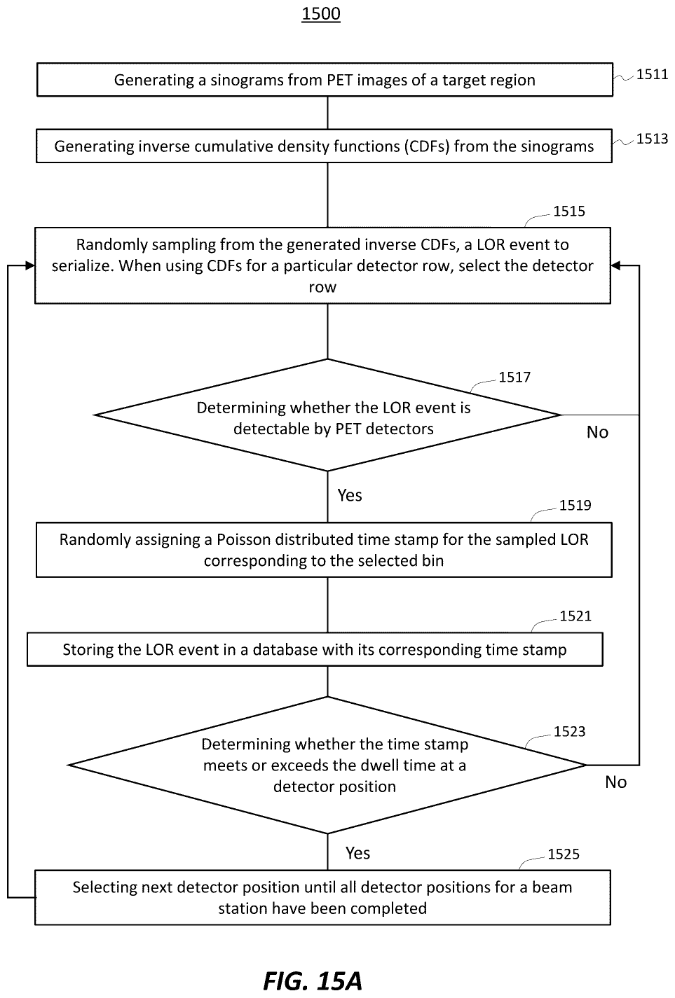

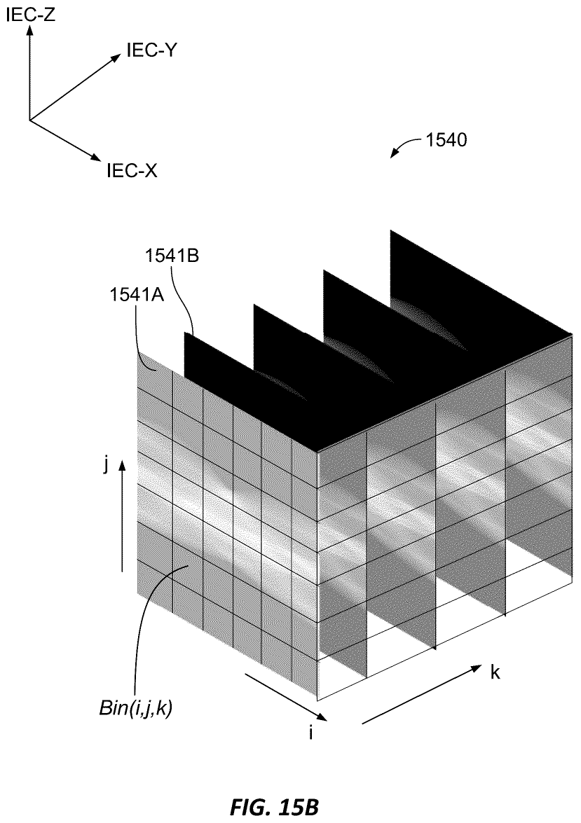

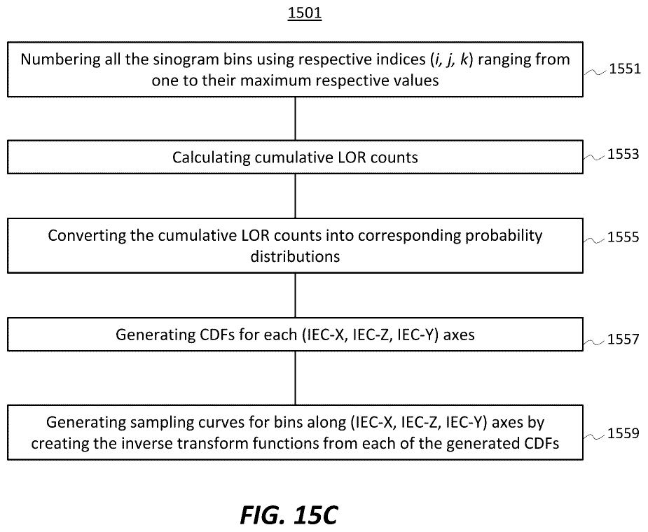

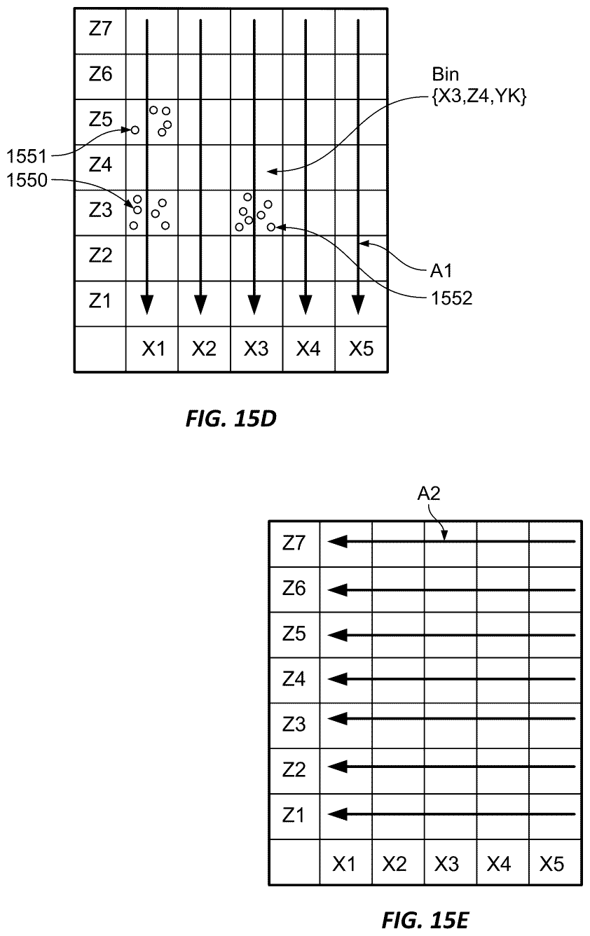

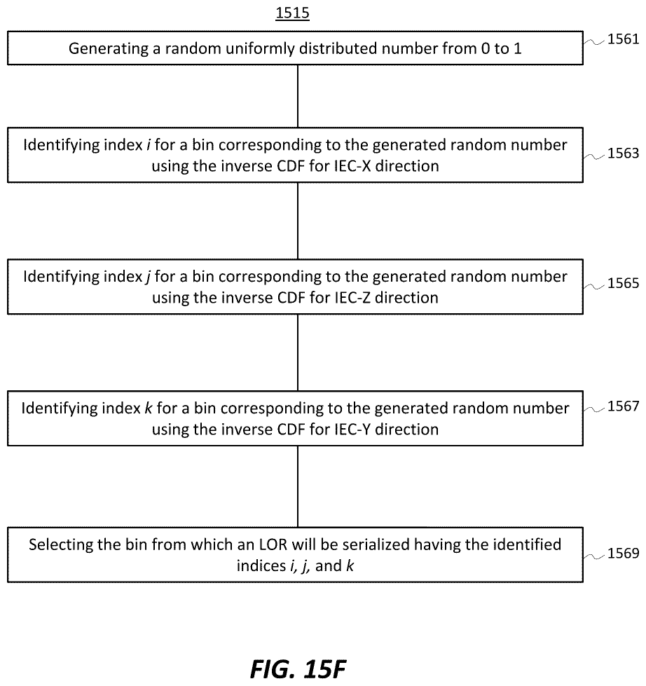

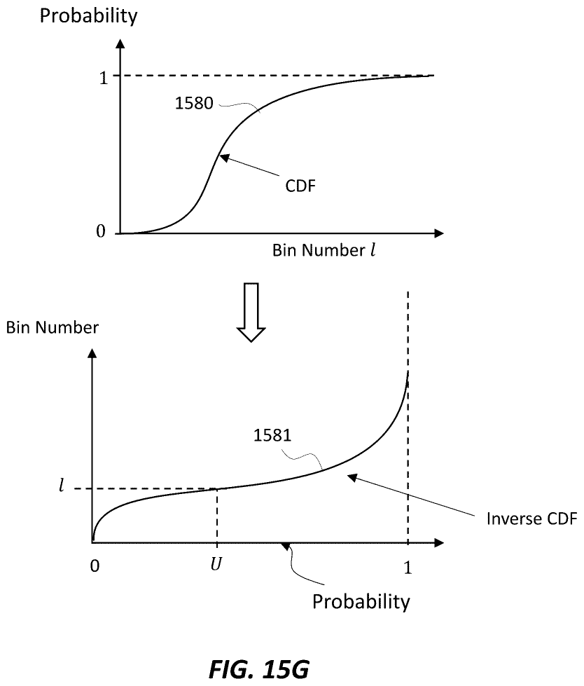

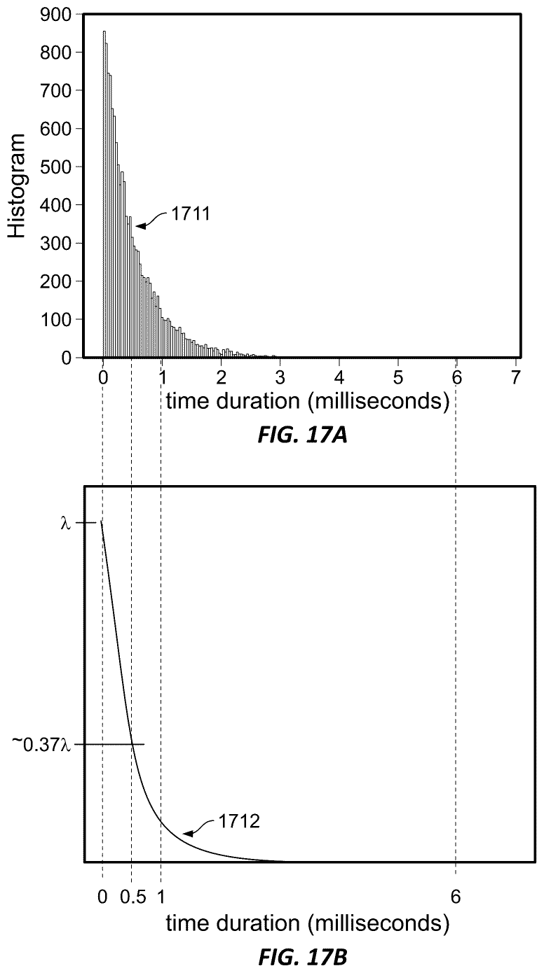

The method 1400 may also include generating 1423 synthetic list mode data for each beam station by serializing the LORs of the second sinogram. Serializing LORs of a beam station sinogram may include resampling the sinogram bins into random events (e.g., see resampling 1447, as shown in FIG. 14B). The resampling 1447 may include transforming the sinogram bins into a set of random LOR events by using inverse transform sampling, as further described below in relation to FIG. 15A. Further, generating 1423 list mode LOR data includes assigning a time stamp (see assigning time stamp 1449, as shown in FIG. 14B) to each event (LOR). The time stamps are randomly assigned and follow an exponential probability distribution (associated with a Poisson probability distribution), as further described below in relation to FIGS. 17A and 17B. Finally, method 1400 may include an optional step 1425 of re-binning list mode LOR data to generate a simulated sinogram (e.g., re-binning 1451 is shown in FIG. 14B resulting in a simulated sinogram 1460) which simulates a typical sinogram that can be obtained during a BgRT PET imaging process. Re-binning 1425 includes associating a sinogram data point with a sinogram bin for each LOR in the list mode LOR data. Herein, a sinogram bin is a region in a sinogram corresponding to a group of similar LOR data points). In some variations, the simulated sinogram may optionally be back projected to PET images that simulate the PET images that may be acquired on a BgRT PET imaging system. These simulated PET images may be analyzed, as described above, to evaluate whether BgRT would be suitable for a patient. In some variations, the steps 1417-1425 may be repeated for each motion phase.

One variation of a method for simulating LORs from a scanner different from the original image may comprise modifying the LORs from the original image to include the effects of scatter, detector efficiency, attenuation, and limitations on the field-of-view. For example, a scattering filter (scatter 1446a, as shown in FIG. 14B), and/or filters associated with a detector efficiency 1446b and field of view corrections 1446c may be applied to LORs from the original image. Additionally, any attenuation correction factors (>1) that were used in the original scanner may be filtered. In some variations, attenuation may be corrected by modifying the conversion to counts. Examples of these filters (1446A-1460D) and compensatory effects are depicted in FIG. 14B. In some variations, LORs that are not within the field of view of the second scanner may be rejected. In some variations, other scatter and random events may be applied directly to the original image.

FIG. 15A shows an example method 1500 of generating list mode LOR data using sinograms from one or more PET images. The method includes generating 1511 diagnostic sinograms from a diagnostic PET image of a target region (or from a computer-generated phantom as discussed above). The diagnostic sinograms may be divided into sections (sinogram bins Bin(i, j, k)) with each bin containing a range of angles Θᵢ=θᵢ±dθ, and range of normal distances (offsets) Sⱼ=sⱼ±ds and a given slice or, in some variations, detector row k (an example detector row k is shown in FIG. 2D). An example set of diagnostic sinograms 1540 is shown in in FIG. 15B. The diagnostic sinograms 1540 include sinogram slices 1541A, 1541B, and so on, with each slice corresponding to the particular detector row k located along the IEC-Y direction. Each sinogram slice is divided into bins as indicated by Bin(i, j, k) in FIG. 15B. The LORs in a particular sinogram bin Bin(i, j, k) have about the same angles θᵢ and about the same normal distances sⱼ and the same detector row k. therefore we can say that Bin(i, j, k) corresponds to LOR(i,j) in row k. Thus, each sinogram bin represents LOR events detected at a corresponding angle θᵢ (e.g., detector position) and offset Sⱼ.

The method 1500 includes generating 1513 inverse cumulative probability density function(s) (CDF(s)) from the PET sinograms (e.g., diagnostic sinograms 1540). The CDFs may be obtained from a histogram of LOR counts from sinogram bins.

In one variation, a method for generating a CDF from a histogram of LOR counts may comprise generating (e.g., plotting) a histogram that indicates the number of counts cᵢ for each sinogram bin Bin(i, j, k). The number of counts c_{i,j,k} may be converted to probabilities p_{i,j,k} by dividing the number of counts by a total count T of all the LORs in all bins, T=Σ_{i=1,j=1,k=1}^{Max(i),Max(j),Max(k)} L(i, j, k), then, p_{i,j,k}=c_{i,j,k}/T. The probability p_{i,j,k} indicates a probability of having one count in the sinogram Bin(i, j, k). In one implementation, bins Bin(i, j, k), may be renumbered sequentially as Bins(l). Alternatively, bins may be renumbered sequentially for each value k (e.g., each sinogram slice, such as 1514A may have sequentially numbered bins Bins(l).) The CDF, for the sinogram, F(l) then may be generated by summing the probabilities F(l)=Σₘ₌₁^{l}pₘ. The generated CDF may then be inverted, for example, using any inverse transfer function and/or by switching the x and y values for each point on the CDF, an example of which are depicted in FIG. 15G.

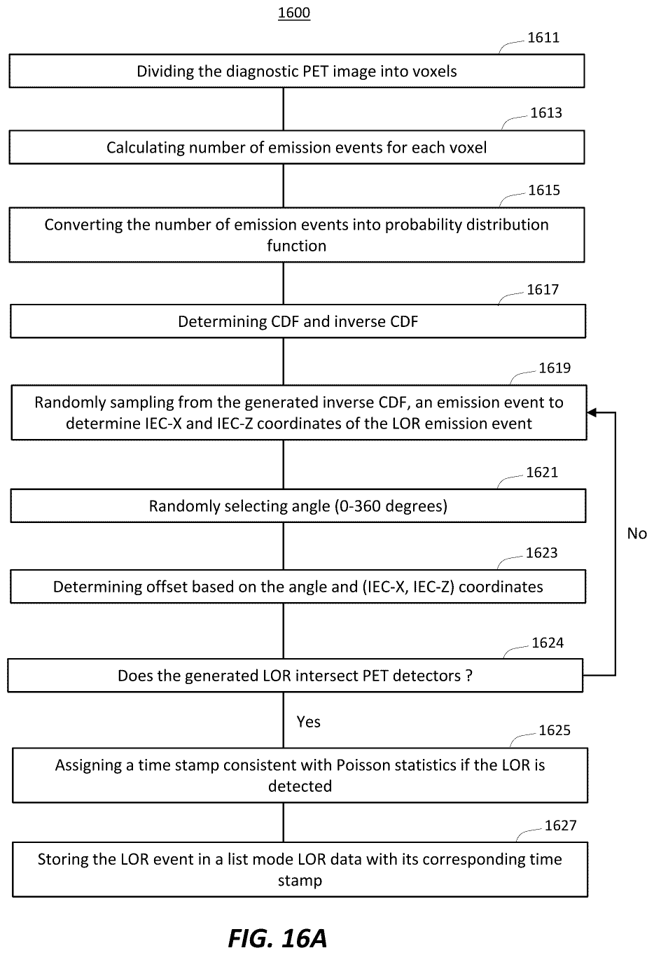

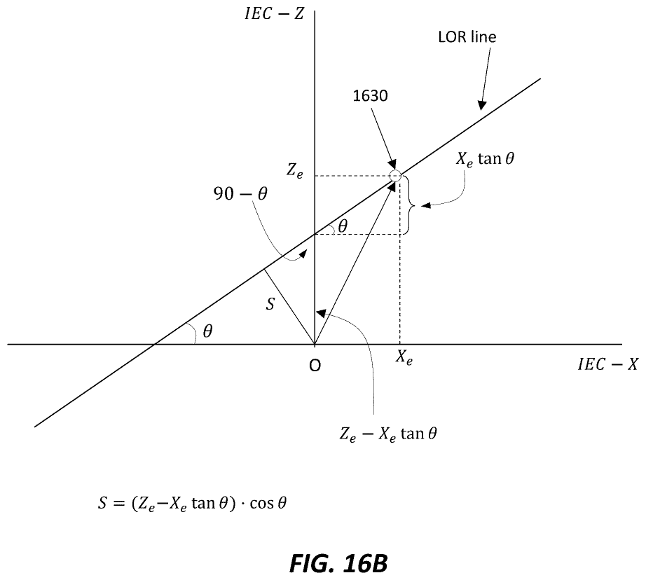

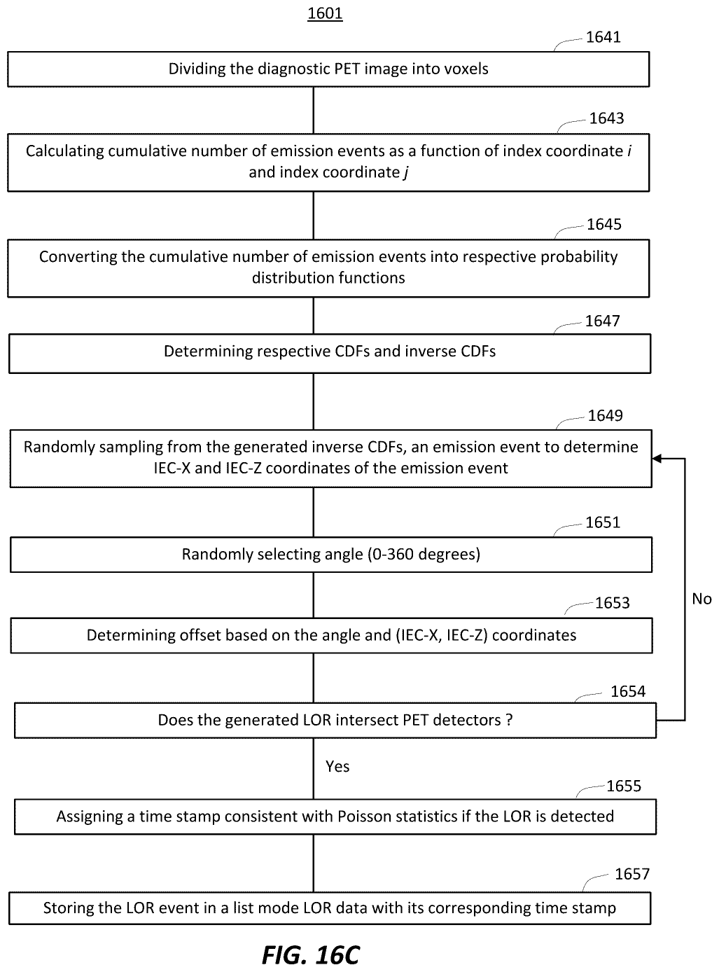

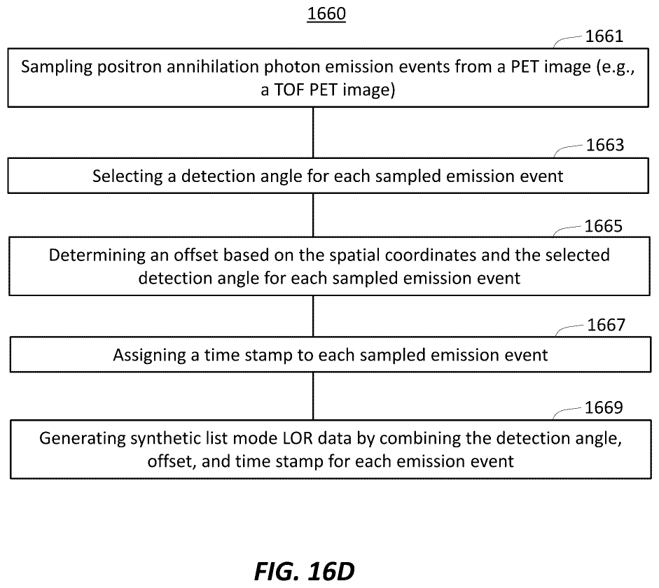

FIG. 15G shows an example cumulative distribution function 1580 as a function bin numbers l (note that bin numbers l are not necessary always associated with sinogram bins, but, in some variations, may be associated with bins (voxels) in a physical space, as further described below in relation to FIG. 16A). Note that CDF 1580 is equal to one at the last bin number, indicating that all the probabilities add up to one (i.e., Σᵢ₌₁^{m}, cₗ/T=1). Further, FIG. 15G shows an inverse CDF 1581, which provides a mapping between sinogram bin numbers and an interval of probability values [0,1]. Inverse CDF 1581 is used for sampling a sinogram bin number l into which an LOR point is being recorded (i.e., into which a count of LOR is added). Such sampling is achieved by first randomly selecting a number U on an interval [0,1], and then using inverse CDF 1581 to obtain the sinogram bin number l for recording the LOR count. The process is repeated until a sufficient number of LORs are sampled. As described above, the number U can be randomly sampled from the interval [0,1] based on a uniform probability distribution.

In an alternative implementation, for example, when there are relatively low LOR counts for each sinogram bin (e.g., less than a few hundred LORs for each sinogram bin, less than a few tens of LORs for each sinogram bin), CDFs may be obtained as indicated by a method 1501, as shown in FIG. 15C. In an example implementation, the method 1501 may correspond to step 1513 of the method 1500.

The method 1501 includes numbering 1551 all the sinogram bins using respective indices (i, j, k) ranging from one to their maximum respective values (Max(i), Max(j), Max(k)). For example, a sinogram bin may be numbered as Bin(i, j, k). Further, the method 1501 includes calculating 1553 cumulative LOR counts as functions of respective indices (i, j, k). Such cumulative LOR counts are referred to as X(i), Z(j), and Y(k) LOR cumulative counts indicating that these LOR cumulative counts are accumulated along respective IEC directions X, Z, and Y. In some variations, the LOR cumulative counts X(i), and Z(j) are calculated for a particular index k, corresponding to a particular row k of detectors, and are referred to as X(i; k) and Z(j; k).

The cumulative LOR counts may be calculated in various ways. In one implementation, the LOR cumulative counts X(i) may be calculated by summing LOR counts L(i, j, k) for each sinogram bin Bin(i, j, k) over two other indices, such as j, and k, as X(i)=Σ_{j=1,k=1}^{Max(j),Max(k)} L(i, j, k). Similarly, Z(j)=Σ_{i=1,k=1}^{Max(i),Max(k)} L(i, j, k), and Y(k)=Σ_{i=1,j=1}^{Max(i),Max(j)} L(i, j, k). Note, that cumulative LOR counts X(i), Z(j), and Y(k) correspond to histograms of LOR counts for each respective IEC directions IEC-X, IEC-Y, and IEC-Z.

In another implementation, when the LOR cumulative counts X(i; k), and Z(j; k) are calculated for a particular index k these cumulative LOR counts X(i; k) and Z(j; k) may be calculated by first summing LOR counts along a respective index j or i, for a sinogram slice at a particular index value k. For instance, FIG. 15D shows schematically bins {Xᵢ, Zⱼ} (e.g., a bin {X3,Z4} is shown in FIG. 15D) for a particular sinogram slice (similar to slice 1541A as shown in FIG. 15B) characterized by a given index k. Each bin {Xᵢ, Zⱼ} contains LOR counts (e.g., LOR counts 1550-1552). In the example implementation, as indicated in FIG. 15D by arrows A1, LOR counts L(i, j, k) at each bin Bin(i, j, k) may be summed as X(i; k)=Σⱼ₌₁^{j=Max(j)} L(i, j, k), to result in a number of LOR counts X(i; k) as a function of index i, for a particular value of k. Herein, as discussed before, Max(j), is the maximum j index. Similarly, as indicated in FIG. 15E by arrows A2, LOR counts L(i, j, k) at each bin Bin(i, j, k) may be summed as Z(j; k)=Σᵢ₌₁^{i=Max(i)} L(i, j, k), to result in a number of LOR counts Z(j; k) as a function of index j, for a particular value of k. Herein, as discussed before, Max(i), is the maximum i index. We can obtain X(i), Z(j), by summing X(i; k) and Z(j; k) over the slices k, that is X(i)=Σₖ₌₁^{Max(k)} X(i; k) and Z(j)=Σₖ₌₁^{Max(k)} Z(j; k). Finally Y(k)=Σ_{i=1,j=1}^{Max(i),Max(j)} L(i, j, k) as previously stated.

The method 1501 further includes converting 1555 the LOR cumulative count X(i) into corresponding probability distribution functions p_{X}(i), p_{Y}(j), and p_{Z}(k) by simply dividing the LOR cumulative count X(i) by a total number of LOR counts T=Σ_{i=1,j=1,k=1}^{Max(i),Max(j),Max(k)} L(i, j, k). Thus, p_{X}(i)=X(i)/T, p_{Z}(j)=Z(j)/T, and p_{Y}(k)=Y(k)/T. Note, for X(i; k), and Z(j; k) LOR cumulative counts, the probabilities are calculated for each value of k as p_{X}(i; k)=X(i; k)/Σ_{i=1,j=1}^{Max(i),Max(j)} L(i, j; k), and p_{Z}(i; k)=Z(j; k)/Σ_{i=1,j=1}^{Max(i),Max(j)} L(i, j; k).

The method 1501 also includes generating 1557 CDFs for each (IEC-X, IEC-Z, IEC-Y) axes by partially summing corresponding probability distribution functions as, CDF_{X}(i)=Σ_{d=1}^{d=1}p_{X}(d), CDF_{Z}(j)=Σ_{d=1}^{d=j}p_{Z}(d), and CDF_{Y}(k)=Σ_{d=1}^{d=k}p_{Y}(d). Note that for X(i; k), and Z(j; k) LOR cumulative counts, the corresponding CDF_{X}(i; k) and CDF_{Z} (j; k) are calculated as CDF_{X}(i; k)=Σ_{d=1}^{d=i}p_{X}(d; k), CDF_{Z}(j)=Σ_{d=1}^{d=j}p_{Z}(d; k). An example of a CDF plot (for any of the axes) is depicted in the upper plot 1840 of FIG. 15G.

Further, the method 1501 includes generating 1559 sampling curves for bins along (IEC-X, IEC-Z, IEC-Y) axes by creating the inverse transform functions from each of the generated CDFs. The inverse transformation functions (herein also referred to as inverse CDFs) can be generated by reflecting CDFs over the vertical axis following by a 90-degree rotation clockwise. An example of an inverse CDF plot (for any of the axes) is depicted in the lower plot 1841 of FIG. 15G. The inverse CDFs map an interval of 0-to-1 (which may be plotted on a horizontal axis of a CDFs plot) to a bin number (which may be plotted on a vertical axis of the CDFs plot). In some variations, any of CDFs CDF_{X}(i), CDF_{Z}(j), CDF_{Y}(k) may be inverted as described herein to result in the corresponding inverse CDFs denoted respectively as ICDF_{X}(p), ICDF_{Z} (p), or ICDF_{Y}(p) with p ranging between 0 and 1, and respective output being i, j, or k. Similarly, for CDF_{X}(i; k), and CDF_{Z} (j; k), corresponding inverse CDFs are ICDF_{X}(p; k), or ICDF_{Z} (p; k) with p ranging between 0 and 1, and respective output being i, j, for a particular value of index k.

Completion of the method 1501 finishes step 1513 of the method 1500. The method 1500 may then include randomly sampling 1515 from the generated inverse CDFs an LOR event for serialization. Random sampling includes selecting a random number p between 0 and 1 (the random number p is selected using uniform probability distribution) and using the selected random number p as an input to an inverse CDF to generate a corresponding to that CDF index (e.g., index i, j, or k, corresponding to associated ICDF_{X}(i), ICDF_{Z}(j) or ICDF_{Y}(k)) of a bin number. The generated indices i, j, and k are then used to select a particular sinogram bin Bin(i, j, k) which yields an LOR event that can be serialized. For ICDF_{X}(p; k), or ICDF_{Z}(p; k), selecting random number p as an input generates indices i, j for a given value of index k.

In some variations, step 1515 may comprise sub-steps 1561-1569 as shown in FIG. 15F, for example when ICDF_{X}(i), ICDF_{Z}(j) or ICDF_{Y}(k)) functions are used. For example, step 1515 may include generating 1561 a random uniformly distributed number from 0 to 1, identifying 1563 index i for a bin corresponding to the generated random number using the inverse CDF for IEC-X direction (e.g., ICDF_{X}(i)), identifying 1565 index j for a bin corresponding to the generated random number using the inverse CDF for IEC-Z direction (e.g., ICDF_{Z}(j)), identifying 1567 index k for a bin corresponding to the generated random number using the inverse CDF for IEC-Y direction (e.g., ICDF_{Y}(k)), and selecting 1569 the bin from which an LOR will be serialized having the identified indices i, j, and k.

The method 1500 includes determining 1517 whether the sampled LOR event is detectable by BgRT radiotherapy system PET detectors (e.g., the LOR event may not be detected by the BgRT radiotherapy system PET detector if the PET detector is positioned such that gamma ray associated with the LOR event does not reach the PET detector). This is done by plotting the sampled LOR in the geometry of the BgRT system and verifying that LOR intersects both detectors for the given firing angle. If the LOR event is determined not to be detected by the BgRT radiotherapy system PET detectors (step 1517, No), the method 1500 proceeds back to step 1515. Alternatively, if the LOR event is determined to be detected by the BgRT radiotherapy system PET detectors (step 1517, Yes), the method 1500 proceeds to randomly assigning 1519 a Poisson distributed time stamp for the sampled LOR corresponding to the selected bin. Further details of assigning the time stamp using a Poisson distribution are described below in relation to FIGS. 17A and 17B.

Further, the method 1500 includes storing 1521 the sampled LOR event in a database with its corresponding time stamp, and determining 1523, based on the assigned time stamp, whether the time stamp meets or exceeds the dwell time at a firing position. If the time stamp does not meet or exceed the dwell time at a given detector position (step 1523, No), the method 1500 proceeds back to step 1515. Alternatively, if the time stamp meets or exceeds the dwell time at the given detector position (step 1523, Yes), the method includes selecting 1525 a next detector position (e.g., position lpos, as described above) until all detector positions for a beam station have been completed.

Methods 1300-1500 relate to the generation of synthetic list mode LOR data using a diagnostic sinogram obtained from a diagnostic PET imaging systems (or a sinogram of a computer-generated PET image of a virtual phantom). When image is generated using data acquired by TOF PET detectors, the diagnostic PET image may not be generated by filtered back projection of the diagnostic sinogram and may not include errors associated with such projection procedures. Diagnostic PET images obtained using TOF PET detectors may record emission events at various voxels in a physical space (herein voxel is referred to as a small volume in a physical space) described by IEC coordinates. For a TOF PET image, coordinates IEC-X, IEC-Z, and IEC Y are known for the location of the annihilation event. However, the angle θ and the offset S for an LOR corresponding to the annihilation event may not be known. Further, a time stamp for the annihilation event may not be known. In some variations, the angle at which a pair of gamma rays is emitted has a uniform distribution, and the uniform distribution can be used to randomly sample angle between 0 and 360, as further described below in relation to FIGS. 16A and 16C.

FIG. 16A is a flowchart representation of one variation of a method for generating synthetic list mode LOR data from a PET image generated from PET data acquired using TOF PET detectors. In one variation, the diagnostic PET image may represent a two-dimensional image and may be taken at an IEC-Y location corresponding to a particular row of detectors (e.g., for a row k of detectors, as shown in FIG. 2D). The method 1600 may include dividing 1611 the diagnostic PET image into pixels or voxels V(i, j). In one variation, index i may indicate the voxel index coordinate along direction IEC-X, and index j may indicate the voxel index coordinate along direction IEC-Z. Note that voxels V(i, j) may partition the two-dimensional diagnostic PET image into small two-dimensional areas. While method 1600 is described in the context of generating list mode data starting with a TOF-PET image, this method may also be used with any PET image where each pixel or voxel of the image is represented by a number of LOR counts or emission events (e.g., the intensity at a pixel or voxel of the PET image correlates to a number of LOR counts or positron annihilation photon emission events).

The method 1600 includes calculating 1613 a number of emission events E(i, j) (i.e., positron annihilation photon emission events) for each voxel V(i, j). Further, the method 1600 includes converting 1615 the number of emission events into a probability distribution function. In an example implementation, the number of emission events E(i, j) may be first renumbered sequentially with a single index l, such that for each pair i, j there is a unique index l. For example, the number of emission events E(i, j) may be renumbered in a column-major order or a row-major order, such that E(i, j)=E (l). Further, the total number of emission events T_{E}=Σ_{i=1,j=1}^{Max(i),Max(j)} E(i, j) is used to obtain a probability distribution function p_{E}(l)=E (l)/T_{E} (here, as before, Max(i) and Max(j) are maximum values of respective indices i, and j).

Further, the method 1600 includes determining 1617 CDF from the probability distribution function obtained in step 1615. In an example implementation the CDF (1) is obtained as CDF_{E}(l)=Σ_{d=1}^{d=1}p_{E}(d). Additionally, the method 1600 includes generating an inverse CDF by reflecting CDF over the vertical axis following by a 90-degree rotation clockwise. The inverse CDF (ICDF_{E}(p)) maps an interval of 0-to-1 (which may be plotted on a horizontal axis of a ICDFs plot) to a voxel number l (which may be plotted on a horizontal axis of a ICDFs plot).

The method 1600 includes randomly sampling 1619 from the generated ICDF_{E}(p) an emission event for serialization. Random sampling includes selecting a random number p between 0 and 1 (the random number p is selected using uniform probability distribution) and using the selected random number p as an input to ICDF_{E}(p) to generate a corresponding to that bin index l which maps to two unique indices i, and j. The generated indices i, and j, are then used to select a particular voxel V(i, j) in which the emission event is determined to occur. Selecting voxel V(i, j) determines physical coordinates IEC-X and IEC-Z of the emission events (e.g., the physical coordinates IEC-X and IEC-Z may be selected to be coordinates of a center of voxel V(i, j) or coordinates of a random point within voxel V(i, j)). In variations where the diagnostic PET image is a 3-D PET image, method 1600 may also comprise randomly sampling from the generated inverse CDF, an emission event to determine the IEC-Y coordinates of the LOR emission event.

The method 1600 may further include randomly selecting 1621 an angle θ for the LOR in a range of 0 to 360 degrees. Once the coordinates IEC-X and IEC-Z are determined, as well as angle θ the offset S can also be determined, as indicated in step 1623 or method 1600. For example, FIG. 16B shows, origin O, IEC-X coordinate Xₑ, IEC-Z coordinate Zₑ, and an LOR line passing through emission event indicated by point 1630. The LOR line is directed at an angle θ, as shown in FIG. 16B. Further, the offset S can be calculated as S=(Zₑ−Xₑ·tan θ)·cos θ.

Method 1600 may comprise determining 1624 whether the generated LOR is detectable by two opposing PET detectors. In some variations, determining 1624 whether the generated LOR is able to be detected by the PET detectors may include determining whether the LOR intersects with the PET detectors (or, in other words, whether the PET detectors are in the path of the LOR). The LOR path may be determined by plotting the calculated angle and offset of the LOR in image space and determining which detectors (if any) are in the LOR path. If the LOR intersects two detectors, it is assumed that the imaging system has detected the LOR and that the LOR can be counted in the list mode data.

If the LOR characterized by angle θ and offset S is detectable by the PET detectors, method 1600 may include assigning 1625 a time stamp based on a Poisson probability distribution (as further described below).

The method 1600 further includes storing 1627 the LOR event in a list mode LOR data with its corresponding time stamp.

FIG. 16C is a flowchart representation of another variation of a method for generating synthetic list mode LOR data from a PET image generated from PET data acquired using TOF PET detectors. The method 1601 may be used when the number of emission events in each voxel is relatively small (e.g., less than a few hundred emission events per each voxel, less than a few tens of emission events per each voxel). Steps 1641 of the method 1601 may be the same as step 1611 of the method 1600. Further, the method 1601 includes calculating 1643 cumulative number of emission events as a function of index coordinate i and index coordinate j. For instance, the cumulative emission events function E_{X}(i) can be calculated as E_{X}(i)=Σⱼ₌₁^{Max(j)} E(i,j), where Max(j) is a maximum j index. Similarly, cumulative emission events function E_{Z}(i) can be calculated as E_{Z}(j)=Σᵢ^{Max(i)} E(i,j), where Max(i) is a maximum i index.

Using the cumulative emission events functions E_{X}(i) and E_{Z}(j), the method 1601 includes converting 1645 the cumulative number of emission events E_{X}(i) and E_{Z}(j), into respective probability distribution functions as p_{EX}=E_{X}(i)/T_{E} and p_{EZ}=E_{Z}(j)/T_{E} (note that Σᵢ₌₁^{Max(i)} p_{EX}=1, and that Σⱼ₌₁^{Max(j)} p_{EZ}=1, as expected).

Further, the method 1601 includes determining 1647 respective CDF_{X}(i) and CDF_{Z}(j) from the probability distribution functions p_{EX} and P_{EZ} obtained in step 1645. In an example implementation the CDF_{X}(i) and CDF_{Z}(j) are obtained as CDF_{X}(i)=Σ_{d=1}^{d=i} p_{EX} (d) and CDF_{Z}(j)=Σ_{d=1}^{d=j} p_{EZ}(d). Additionally, the method 1600 includes determining an inverse ICDF_{EX}(p) and ICDF_{EZ}(p) by reflecting the respective CDFs over the vertical axis following by a 90-degree rotation clockwise. The general process of converting a cumulative probability function into an inverse CDF is described above, for example, in FIG. 15C. Examples of CDF and the corresponding inverse CDF are depicted in FIG. 15G. The ICDF_{EX}(p) maps an interval of 0-to-1 to an index number i, and the ICDF_{EZ}(p) maps an interval of 0-to-1 to an index number j. Once indices i, j, are determined these indices are then used to select a particular voxel V(i,j) in which the emission event is determined to occur. Selecting voxel V(i,j) determines physical coordinates IEC-X and IEC-Z of the emission events. After completing step 1649, the method 1601 may proceed to step 1651-1657, which may be the same as respective steps 1621-1627, of the method 1600.

FIG. 16D depicts one variation of a method 1660 for converting a PET image into synthetic lines-of-responses (LORs). The PET image may be a diagnostic PET image generated using TOF PET, and/or a computer-generated PET image of a virtual phantom, where the intensity of each pixel of the PET image is correlated to a number of emission events that have occurred at the spatial location of that pixel. Method 1660 may include sampling 1661 positron annihilation photon emission events from a PET image, selecting 1663 a detection angle for each sampled emission event, determining 1665 an offset based on the spatial coordinates and the selected detection angle for each sampled emission event, assigning 1667 a time stamp to each sampled emission event, and generating 1669 synthetic list mode LOR data by combining the detection angle, offset, and time stamp for each emission event. The initial PET image may be a TOF PET image or any PET image where an intensity of each pixel correlates to a number of emission events having spatial coordinates that correspond to a location of that pixel. In some variations, sampling the emission events may include converting the number of emission events into a probability distribution function, determining a cumulative distribution function (CDF) and an inverse CDF, and randomly selecting emission events from the generated inverse CDF (as described above in reference to FIG. 15G). Selecting the detection angle may include randomly selecting an angle in a range of 0 degrees to 360 degrees. The spatial coordinates of a pixel and the corresponding emission events may include coordinates in IEC-X and IEC-Z, and the offset may be determined using the IEC-X coordinate, IEC-Z coordinate, and the selected detection angle. In some variations, method 1660 may further comprise determining whether an LOR corresponding to an emission event (with its spatial coordinates, selected detection angle, and determined offset) intersects with PET detectors of a PET imaging system before assigning a time stamp to the emission event. In some variations, assigning the time stamp for each emission event may include selecting time intervals between emission events according to Poisson statistics, as described further below.

As described above, methods 1500, 1600, 1601, and 1660 include assigning a time stamp for a sampled LOR based on a Poisson probability distribution function which is related to the exponential distribution function. The probability of an emission event occurring after a previous emission event in time t is given by an exponential CDF as P(t)=1−exp (−λ·t), which is obtained by integrating an exponential probability distribution function ƒ(t)=λ·exp (−λ·t). The exponential probability distribution function ƒ(t) may be obtained from a histogram of emission events recorded as a function of time. An example histogram 1711 of emission events is shown in FIG. 17A. The histogram 1711 indicates number of LOR counts as a function of time (time in milliseconds) is indicated on a horizontal axis). For example, during a first millisecond an average of about 350 counts are recorded. The histogram 1711 may be converted into a probability distribution function by dividing LOR counts at each time point by a total number of counts. The example exponential probability distribution function 1712 is shown in FIG. 17B, and is given by expression ƒ(t)=λ·exp (−λ·t), where λ is a decay rate indicating how quickly probability distribution function 1712 decays with time. For example, the probability distribution function 1712 decays by a factor of e (factor of about 2.8) in about half of millisecond, indicating that exp (−1)=exp (−λ·0.5·10⁻⁶), or that λ˜2000 [1/s]. The CDF P(t)=1−exp (−λ·t) indicates a probability of a duration of time needed for a LOR count to occur after a previous count was recorded. For instance, about sixty percent of the time the duration between two subsequent counts may be less than one half of a millisecond (P(0.5 ms)=1−exp (−1)=0.63). The expected value of the probability distribution function 1712 is 1/λ and is estimated to be about half of a millisecond. Therefore, for collecting 10,000 LORs, one on average requires about 10,000/2, or 0.05 seconds. This is similar to a dwell time during the BgRT procedure at each beam station. For instance, during a BgRT procedure, data may be collected for about a few seconds at each beam station, and there may be 4 passes for each beam station resulting in total collection time of about a few tens of seconds per beam station. For instance, in 20 seconds, a number of LORs collected is equal to 20.2=20.2000=40,000 LORs.

In some variations, the time interval between LOR events or counts may be generated by selecting a random number p between 0 and 1, and identifying the time interval on the CDF corresponding to the value of p. Alternatively, or additionally, the CDF P(t)=1−exp (−λ·t) may be converted into an inverse CFD (ICDF) as

$$ICDF ( t ) = - ( \frac{1}{λ} ) ln ( 1 - p ) ,$$

with p ranging from 0 to 1. The ICDF (p) maps an interval of 0-to-1 to a time difference Δt(k−1; k) between two successive emission events Eₖ₋₁ and Eₖ. The time stamp tₛ(k) for event Eₖ may be obtained by summing all the time differences as tₛ(k)=Σ_{d=1}^{d=k}Δt(d−1; d). When time stamp tₛ(k) exceeds the dwell time at a firing position, all LORs may be collected and a detector position may be changed (e.g., a gantry containing the PET detectors may move to a new firing angle position lpos). In an example implementation the gantry may spend a few milliseconds (e.g., 1-20 milliseconds) at each firing angle position lpos.

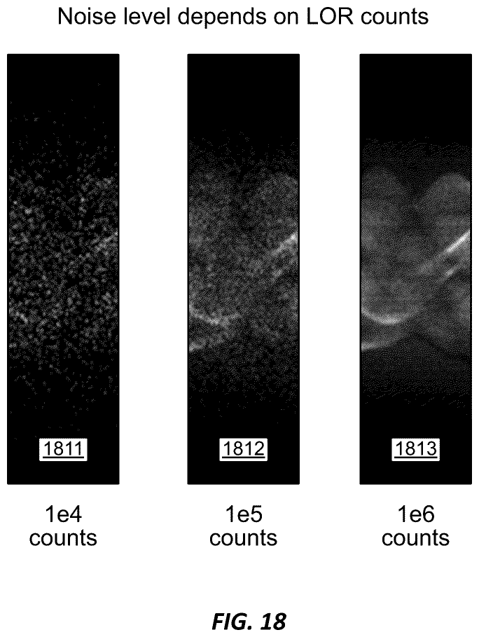

The simulated (e.g., synthetic) LORs, time stamps, detector positions etc. can all be assembled in a “list mode” table. The synthetic list mode LOR data can then be used to generate sinograms that may include artifacts associated noisy or imperfect (e.g., incomplete) LOR sampling. FIG. 18 shows sinograms 1811-1813 with increasing LOR counts. Note that the noise is most evident for the sinogram 1811 exhibiting the lowest LOR counts. In some variations, the sinograms generated from the synthetic list mode LOR data may be back projected and used to compare images generated by a TOF-PET imaging system and images generated by a non-TOF PET imaging system.

In some variations, when radioactivity emission rate is sufficiently low the number of LOR counts in each sinogram bin may be low (e.g., a few tens of LOR counts). Such low number of LOR counts may result in an artificial “quantization noise” when the diagnostic noiseless sinogram is converted to a second sinogram, as described in step 1419 of the method 1400. This quantization noise is analogous to “digitization noise” in A/D converters. The “noisy” fluctuating count rate will translate to a noisy probability distribution, followed by a noisy CDF and ultimately such noise may be introduced into the synthetic sampled LORs.

There are several ways around this problem including artificially increasing the activity (and thus counts), then effectively reducing the imaging time to compensate. For example, some variations may comprise increasing the activity by 10× the number of LOR counts and sampling the inverse transform for a shorter period of time (e.g., for a tenth of the period of time, 1 ms rather than 10 ms). This will create a much “smoother” CDF from which to select a bin to serialize.

In some variations, the process of modifying the counts for each firing position of each beam station further includes modifying the counts based on a selected motion trajectory for the target region (e.g., based on a breathing or peristaltic motion). The motion trajectory is associated with a measure of a motion or deformation of tissues (e.g., tissues of body organ or collection of body organs) associated with the anatomy. For each time point of the motion trajectory, a corresponding sinogram may be generated based on an associated PET image data for that time point, and that sinogram may be converted to counts as described above, thus, accounting for motion of the anatomy.

The motion trajectory may correspond to any suitable motion of the anatomy. For instance, when the motion trajectory corresponds to a breathing cycle (e.g., the breathing cycle of a person), such motion trajectory is referred to as a breathing motion trajectory. Further, when the motion trajectory corresponds to a cardiac cycle (e.g., the cardiac cycle of a person), such motion trajectory is referred to as a peristaltic motion trajectory or cardiac motion trajectory. Alternatively, the motion trajectory may be a user-defined motion trajectory (e.g., the motion trajectory where a specific movement of the anatomy is specified) as a function of time.

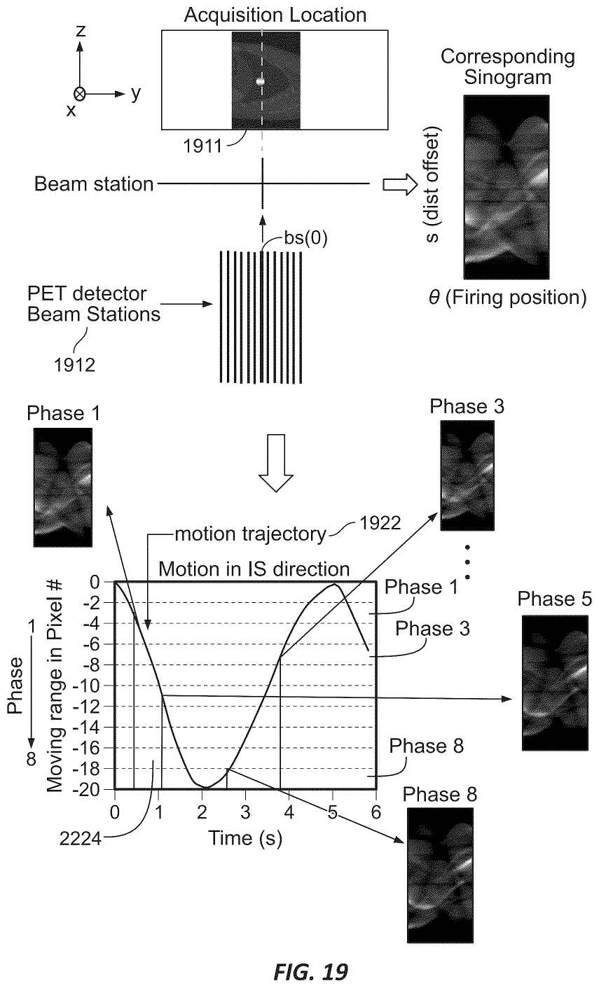

FIG. 19 shows an example process of acquiring data from a phantom 1911 when various regions of the phantom 1911 move relative to each other (e.g., such movement can correspond to movements of lungs during a breathing of a person or movements of heart muscles) or deform (e.g., change shape). In some variations, phantom 1911 is associated with multiple PET images acquired from a patient at different times (e.g., every few milliseconds for a duration of a few seconds or minutes) to capture the movements of body tissues. Alternatively, phantom 1911 may be an object engineered for testing PET image scanners and is configured to have various regions capable of motion. Alternatively, phantom 1911 may be a computer-generated data (e.g., data which resembles PET images from a patient taken at different points in time during a breathing cycle of a patient or during a cardiac cycle of the patient). In some variations, computer-generated PET images of a moving virtual phantom may be used to model patient motion.

In one variation, the PET images corresponding to a breathing cycle (or a cardiac cycle) may be separated into distinct phases based on movement of the tissues associates with the PET images. In one example, the phases may be separated from each other by a constant duration of time. Alternatively, the phases may correspond to various stages of the breathing cycle determined based on moving range of pixels forming PET images and exemplified by a motion trajectory 1922 as shown in FIG. 19. The specific phases may then be sampled from this motion trajectory 1922 by subdividing an ordinate (vertical) axis of the motion trajectory 1922 into segments as indicated by dashed lines 1924. For example, sinograms corresponding to phases 1, 3, 5, and 8, of the motion trajectory 1922 are shown in FIG. 19. The sinograms generated for each bin may be converted into synthetic list mode LOR data using any of the methods described herein. The synthetic list mode LOR data generated using any of the methods described above may be used to test delivery algorithms for BgRT and evaluate whether those delivery algorithms would provide the prescribed dose of radiation to target regions while adhering to dose limitations of surrounding tissue. In variations where diagnostic PET images are acquired over time (i.e., 4D PET images), these may be converted into synthetic list mode LOR data and then converted into synthetic sinograms that are back-projected into simulated PET images. These PET signal of these simulated PET images may be evaluated using any of the metrics described above to determine whether BgRT is suitable for a patient.

While different variations have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the example inventions described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive variations described herein. It is, therefore, to be understood that the foregoing variations are presented by way of example only and that, within the scope of the appended claims and equivalents thereto; inventive variations may be practiced otherwise than as specifically described and claimed. Inventive variations of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described systems and methods can be implemented in any of numerous ways. For example, at least some methods of the present technology may be implemented using hardware, firmware, software, or a combination thereof. When implemented in firmware and/or software, the firmware and/or software code can be executed on any suitable processor or collection of logic components, whether provided in a single device or distributed among multiple devices.

In this respect, various aspects described herein may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various examples of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of example inventions as discussed above. Additionally, it should be appreciated that according to one aspect, one or more computer programs that when executed perform methods of the present invention need not reside on a single computer or processor but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in different variations.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, various inventive concepts may be embodied as one or more methods, of which an example has been provided. The acts performed as part of the method may be ordered in any suitable way. Accordingly, variations of the invention may be constructed in which acts are performed in an order different than illustrated, which may include performing some acts simultaneously, even though shown as sequential acts in illustrative variations and examples.

1. A method for determining suitability of biology-guided radiotherapy (BgRT), the method comprising:
converting diagnostic positron emission tomography (PET) imaging data of a tumor to simulated imaging data consistent with images obtained using PET detectors of a BgRT radiotherapy system, the simulated imaging data and the diagnostic PET imaging data representing a PET signal from a tracer;
calculating a first metric indicating a contrast noise ratio for the tumor using the simulated imaging data;
calculating a second metric indicating a PET tracer activity concentration using the simulated imaging data;
calculating a third metric indicating a radiation dose to the tumor using the simulated imaging data; and
determining that BgRT is suitable if a value of at least one of the first, the second, and the third metric is within a range of acceptable values.

2. The method of claim 1, further comprising:
obtaining additional diagnostic PET imaging data;
converting the additional diagnostic PET imaging data to new simulated imaging data consistent with images obtained when performing BgRT;
calculating a new first metric value indicating a contrast normalization signal for a tumor;
calculating a new second metric value indicating a PET tracer activity concentration;
calculating a new third metric value indicating a radiation dose for a volume of the tumor; and
determining that BgRT is suitable if a value of at least one of the new first metric value, the new second metric value, and the new third metric value is within the range of acceptable values.

3. The method of claim 2, wherein the determining the suitability of using the BgRT is further based on a difference between the new first metric value and the first metric value, the new second metric value and the second metric value, and the new third metric value and the third metric value.

4. The method of claim 2, wherein the additional diagnostic PET imaging data is obtained prior to performing a BgRT treatment, the BgRT treatment not forming part of the method.

5. The method of claim 1, wherein the first metric is determined as a difference between a mean signal in a target region <T_{S}> and a mean signal in a background region <Bg> divided by a variance of the signal σ_{Bg} in the background region:
(<T_{S}>−<Bg>)/σ_{Bg}.

6. The method of claim 5, wherein the signal in a target region Tₛ is calculated in a portion of a clinical target volume in which a value of a PET signal is less than a target threshold percent of a peak value of the PET signal as measured in the clinical target volume.

7. The method of claim 6, wherein the target threshold percent is fifty percent.

8. The method of claim 1, wherein the first metric is determined as a median activity concentration of a target region (PTV) divided by a mean signal in a background region <Bg>:
MedianAC[PTV]/<Bg>.

9. The method of claim 8, wherein Bg is calculated over a shell region, the shell region being a portion of a biological targeting zone and not a part of a clinical target volume.

10. The method of claim 1, wherein determining the suitability of using the BgRT comprises determining that:
the contrast normalization signal is above a required threshold for the signal;
the PET tracer activity concentration is above a minimal concentration threshold; and
the determined radiation dose is within a pre-defined dose range.

11. The method of claim 10, wherein the pre-defined dose range is represented by an upper dose-volume histogram (DVH) curve and a lower DVH curve of a bounded DVH.

12. The method of claim 1, wherein, when the suitability of using BgRT is not indicated, obtaining an additional diagnostic PET imaging data using a different type of PET tracer than a type of PET tracer that is used for obtaining the diagnostic PET imaging data.

13. The method of claim 1, wherein, a first metric is further verified by obtaining visual representation of the tumor using CT imaging.

14. The method of claim 1, wherein the radiation dose comprises a function determining acceptable radiation doses for a given volume fraction of a tumor tissue.

15. The method of claim 1, further comprising converting the simulated imaging data to single line-of-response (LOR) data between a pair of detector elements.

16. The method of claim 1, further comprising generating a BgRT plan, the BgRT plan including:
an identified target region; and
firing filters that convert PET imaging data into a radiation fluence map that results in the prescribed dose being delivered to the identified tissue.

17. The method of claim 1, wherein converting the diagnostic PET imaging data to the simulated imaging data consistent with images obtained using PET detectors of a BgRT radiotherapy system comprises:
calibrating sensitivity of the PET detectors of the BgRT radiotherapy system;
generating a sinogram based on the PET imaging data, wherein the generating includes correcting for an attenuation using computer tomography (CT) data;
converting the sinogram to expected counts per sinogram-bin;
modifying the expected counts based on parameters of the BgRT radiotherapy system, wherein the parameters include at least the sensitivity of the BgRT radiotherapy system subject to an efficiency of the BgRT radiotherapy system and a time used by the BgRT radiotherapy system for collecting data;
modifying the expected counts by adding noise modeled by Poisson statistics; and
reconstructing the simulated imaging data based on the modified expected counts.

18. The method of claim 17, wherein converting the diagnostic PET imaging data to the simulated imaging data consistent with images obtained using PET detectors of a BgRT radiotherapy system further comprises:
determining the sinogram based on the PET imaging data by modeling photon scatter in a PET detector scintillator.

19. The method of claim 17, wherein the noise modeled by Poisson statistics is based on random coincidences.

20. The method of claim 17, wherein the noise modeled by Poisson statistics is based on random detection events.

21. The method of claim 17, wherein the sinogram is corrected by truncating the sinogram to a field of view that includes the tumor.

22. The method of claim 21, wherein the target field of view has a size of 50 centimeters.

23-25. (canceled)

26. A method for simulating a second PET image based on a first PET image, the method comprising:
converting a first PET image of a target region into a sinogram;
generating list mode data from the sinogram by sampling LORs from the sinogram to include noise characteristics and component characteristics of a PET imaging system and serializing the sampled LORs into a list mode LOR data, with each sampled LOR having a corresponding time stamp; and
generating a second PET image of the target region by filtering and backprojecting the list mode LOR data.

27-51. (canceled)

52. A method for converting a PET image into simulated lines-of-responses (LORs) the method comprising:
generating a sinogram from a PET image of a target region; and
generating a list mode LOR data based on the generated sinogram, wherein the list mode LOR data comprises a list of simulated LORs, and wherein the list of the simulated LORs is generated based on a sample of emission events.

53-65. (canceled)

66. A method for simulating a second PET image based on a first PET image, the method comprising:
converting a first PET image of a target region into a plot that comprises a number of positron annihilation photon emission events for each pixel in a PET image;
sampling emission events from the plot to include noise characteristics and component characteristics a PET imaging system;
generating list mode data from the plot by serializing the sampled emission events by assigning a time stamp to each sampled emission event; and
generating a second PET image of the target region using the list mode data by plotting an intensity level at every pixel that correlates with the number of emission events at that pixel.

67. The method of claim 66, wherein the noise characteristics of PET detectors of the PET imaging system comprise at least one of: photon scatter noise, Poisson noise, attenuation effects, and random photon coincidences.

68. The method of claim 66, wherein the component characteristics of PET detectors comprise at least one of: detection efficiency, detector crystal width, detector acquisition rate, detector resolution, and detector time resolution.

69. The method of claim 66, wherein the list mode data include time stamps corresponding to individual LORs from the sampled emission events.

70. The method of claim 66, wherein the first PET image comprises a plurality of PET images acquired of the target region over time.

71. The method of claim 70, wherein a location of the target region changes with time along a motion trajectory, and wherein the plurality of PET images are obtained for different points in time.

72. The method of claim 71, further comprising:
grouping each of the plurality of PET images into PET image phases based on the location of the target region along the motion trajectory;
for each phase, selecting a representative PET image as the first PET image and generating list mode data for each phase by converting the PET image into a plot comprising a number of positron annihilation photon emission events for each pixel, sampling emission events from the plot, and serializing the sampled emission events by assigning a time stamp to each sampled emission event.

73. The method of claim 72, further comprising generating a sinogram for each phase derived from the list mode data for that phase.

74. The method of claim 71, wherein the motion trajectory of the target region is a breathing motion trajectory.

75. The method of claim 71, wherein the motion trajectory of the target region is a peristaltic motion trajectory.

76. The method of claim 71, wherein the motion trajectory of the target region is a user-defined motion trajectory.

77. The method of claim 66, wherein the list mode data comprises a plurality of emission events, each emission event having a corresponding detection event time stamp and associated coordinates of detectors for detecting an LOR for each emission event.

78. The method of claim 66, wherein the first PET image is a time-of-flight PET image.

79-84. (canceled)
